(19) United States
(12) Patent Application Publication
(10) Pub. No.: US 2023/0020548 A1
(43) Pub. Date: Jan. 19, 2023

(54) ANTI-INTERLEUKIN 1 BETA ANTIBODIES FOR TREATMENT OF SICKLE CELL DISEASE

(71) Applicant: NOVARTIS AG, Basel (CH)

(72) Inventors: Alexandre Yann Guichard, Colmar (FR); Jiri Kovarik, Zürich (CH); Yue Li, Ettingen (CH); Elizabeth Akselrod McNamara, Salem, MA (US); Karin Sigrid Meiser, Weil am Rhein (DE); Stephen John Oliver, Basel (CH); Michael Shi, Chalfont, PA (US)

(21) Appl. No.: 17/783,019

(22) PCT Filed: Oct. 26, 2020
(86) PCT No.: PCT/IB2020/060030

§ 371 Date: Jun. 7, 2022

Related U.S. Application Data
(60) Provisional application No. 62/945,594, filed on Dec. 9, 2019.

(51) Int. Cl. C07K 16/24; A61P 7/00

(52) U.S. Cl. CPC C07K 16/245; A61P 7/00; A61K 2039/505

(57) ABSTRACT

This disclosure relates to use of anti-IL-1β antibodies (e.g., canakinumab or gevokizumab) in a therapy treating, preventing, reducing, or alleviating one or more manifestations and complications in individuals suffering from sickle cell disease (including, e.g., homozygous HbS gene carriers, heterozygotes with sickle-beta-thalassemia with an SCD supporting combination of HbS gene and beta-tha1 gene, an individual with one sickle cell gene and one null allele, or an individual with hemoglobin sickle cell disease), optionally in combination with an additional therapeutic agent. Such manifestations and complications include, but are not limited to: pain, fatigue, hospitalization, poor sleep quality, work or school absences, narcotic use, acute or chronic blood transfusion therapy, acute chest syndrome, bone infarct, avascular necrosis, osteonecrosis, stroke, priapism, infarction of penis, a cardiovascular disorder, growth delay, stunted growth, low body mass index (BMI), low body weight, and organ damage.

### BACKGROUND

Sickle cell disease is a group of inherited red blood cell disorders that affects a considerable percentage of people that come from tropical or subtropical regions where malaria is or was common, or descendants thereof. It is a recessive autosomal genetic blood disorder, and presence of one sickle cell gene in a person enhances fitness against malaria. This has led to an evolutionary selection of people carrying one allele of the gene, in spite of the strong disadvantages for carriers of two alleles.

Approximately 100,000 individuals have sickle cell disease in the USA, and world-wide approximately 300,000 children are born each year who are affected by the disease.

In sickle cell disease, endothelial activation and leukocyte recruitment (caused by reperfusion injury, inflammatory stimuli, infection, and/or hypersensitivity reactions) can lead to flow stasis, intravascular sickling and vaso-occlusion, a hallmark of this disease. Reperfusion injury (caused by vaso-occlusive events) and elevated levels of inflammatory cytokines in this disease are implicated in endothelial activation and inflammation. In transgenic sickle mice, exaggerated inflammatory response to hypoxia-reoxygenation is characterized by enhanced endothelial oxidant generation and leukocyte recruitment in venules (Kaul and Hebbel, J. Clin Invest. 106, 411-420, 2000). While during the hypoxic phase intravascular sickling is enhanced, reperfusion will induce exaggerated inflammatory response and leukocyte recruitment, which promotes flow stasis (Turhan et al. Proc. Natl. Acad. Sci. USA 99(5), 3047-3051, 2002). Hypoxia also induces endothelial cells and leukocytes to express and secrete proinflammatory cytokines such as interleukin-1 beta (IL-1β). Patients who have sickle cell disease show elevated levels of IL-1β, which may, in part, be attributed to recurring ischemia-reperfusion episodes. IL-1β can up-regulate endothelial adhesion molecules via endothelial oxidant generation and activation of nuclear factor-κB (NF-κB). In SCD, IL-1β is implicated in endothelial activation, cell adhesion and inflammation (Natarajan et al., Blood 87, 4845-4852 (1996); Zachlederova and Jarolim, Blood Cells Mol. Dis. 30, 70-81, 2003; Wanderer, J. Pediatr. Hema-tol. Concol. 31(8), 537-538, 2009).

Inflammatory conditions, infections, or hypersensitivity reactions will cause abnormal activation of endothelium and increased leukocyte recruitment in postcapillary venules resulting in sluggish blood flow, increased microvascular transit times, hypoxia, red cell sickling, and vaso-occlusion. Sickle cell disease can lead to a host of manifestations and complications including frequent and debilitating pain, anemia, stunted growth, stroke, and hospitalization.

Hence, effective therapeutic approaches are required to prevent such manifestations and complications in patients who have sickle cell disease.

### SUMMARY

Herein is demonstrated for the first time that use of an anti-interleukin 1beta antibody in patients with human sickle cell disease (e.g., sickle cell anemia) results in significant reductions of serum markers of systemic inflammation, including hs-CRP and total circulating leukocyte counts (e.g., the absolute counts of neutrophils and monocytes, biomarkers of which have been reported to be predictive of long term morbidity and mortality for this patient population). In addition, the use of an anti-interleukin 1beta antibody in patients with human sickle cell disease (e.g., sickle cell anemia) resulted in reduced transcranial Doppler (TCD) velocities. This is particularly significant in that transcranial Doppler velocities (MCA velocities) of more than 200 cm/s are associated with a heightened risk of stroke in patients with sickle cell disease. Further, the use of an anti-interleukin 1beta antibody in patients with human sickle cell disease (e.g., sickle cell anemia) resulted in increases in body weight and body mass index (BMI), ameliorating the growth delays experienced by these patients. Additional benefits include, but are not limited to: reduced pain, fatigue, reduced hospital stays, and a decrease in absence days from school or from work.

Thus, in one aspect of the invention, herein are provided methods of treating a complication or manifestation associated with sickle cell disease in a subject in need thereof, the method comprising administering to the subject a therapeutically effective amount of an antibody or antigen binding fragment that specifically binds to IL-1 beta (e.g., canakinumab or gevokizumab) or any one of the combinations described herein.

Thus, in one aspect of the invention, herein is provided a use of an antibody or antigen binding fragment that specifically binds to IL-1 beta (e.g., canakinumab or gevokizumab) or any one of the combinations described herein, for the manufacture of a medicament for the treatment of a manifestation or complication associated with sickle cell disease in a subject in need of such treatment.

Thus, in one aspect of the invention, herein is provided an antibody or antigen binding fragment that specifically binds to IL-1 beta (e.g., canakinumab or gevokizumab) or any one of the combinations described herein, for use in the treatment of a manifestation or complication associated with sickle cell disease in a subject in need of such treatment.

In one aspect, provided herein are methods of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta, optionally wherein the manifestation or complication is selected from: intravascular inflammation, endothelial dysfunction, pain, fatigue, hospitalization, poor sleep quality, work absences, school absences, narcotic use, acute blood transfusion therapy given for disease severity, chronic blood transfusion therapy given for disease severity, acute chest syndrome, bone infarct, avascular necrosis, osteonecrosis, stroke, cognitive dysfunction, priapism, infarction of penis, a cardiovascular disorder, growth delay, stunted growth, low body mass index (BMI), low body weight, and organ damage.

In some embodiments, the subject is a pediatric patient. In some embodiments, the subject is an adult patient. In some embodiments the patient is 2 years old to 18 years old. In some embodiments the patient is 2 years old to 16 years old. In some embodiments, the patient is 5 years old to 16 years old. In certain embodiments, the subject is 21 years old or older. In some embodiments, the subject has a weight below 40 kg. In some embodiments, the subject has a weight below 40 kg and the therapeutically effective amount of an antibody or antigen binding fragment that specifically binds to IL-1 beta is 2 mg/kg to 4 mg/kg. In some embodiments, the subject has a weight below 40 kg and the therapeutically effective amount of an antibody or antigen binding fragment that specifically binds to IL-1 beta is 2 mg/kg. In some embodiments, the subject has a weight below 40 kg and the therapeutically effective amount of an antibody or antigen binding fragment that specifically binds to IL-1 beta is 4 mg/kg. In some embodiments, the subject has a weight below 40 kg and the therapeutically effective amount of an antibody or antigen binding fragment that specifically binds to IL-1 beta is 4 mg/kg as loading dose and 2 mg/kg as maintenance dose. In some embodiments, the subject has a weight below 40 kg and the therapeutically effective amount of an antibody or antigen binding fragment that specifically binds to IL-1 beta is about 2 mg/kg to about 4 mg/kg. In some embodiments, the subject has a weight below 40 kg and the therapeutically effective amount of an antibody or antigen binding fragment that specifically binds to IL-1 beta is about 2 mg/kg. In some embodiments, the subject has a weight below 40 kg and the therapeutically effective amount of an antibody or antigen binding fragment that specifically binds to IL-1 beta is about 4 mg/kg. In some embodiments, the subject has a weight below 40 kg and the therapeutically effective amount of an antibody or antigen binding fragment that specifically binds to IL-1 beta is about 4 mg/kg as loading dose and about 2 mg/kg as maintenance dose.

In some embodiments, the subject has been diagnosed with sickle cell anemia. In some embodiments, the subject has HbSS or HbS-beta⁰ thalassemia. In some embodiments, the subject has HbSS thalassemia. In some embodiments, the subject has HbS-beta⁰ thalassemia.

In some embodiments, the subject has an hs-CRP level of at least about 1 mg/L or at least about 2 mg/L prior to administration of the antibody or antigen binding fragment thereof. In some embodiments, the subject has an hs-CRP level of at least about 1 mg/L prior to administration of the antibody or antigen binding fragment thereof. In some embodiments, the subject has an hs-CRP level of at least about 2 mg/L prior to administration of the antibody or antigen binding fragment thereof. In some embodiments, the subject has an hs-CRP level of at least about 3 mg/L prior to administration of the antibody or antigen binding fragment thereof. In some embodiments, the subject is a pediatric patient and has an hs-CRP level of at least about 1 mg/L prior to administration of the antibody or antigen binding fragment thereof. In some embodiments, the subject is an adolescent patient and has an hs-CRP level of at least about 1.5 mg/L prior to administration of the antibody or antigen binding fragment thereof. In some embodiments, the subject is an adult patient and has an hs-CRP level of at least about 2 mg/L prior to administration of the antibody or antigen binding fragment thereof.

In some embodiments, the subject has an hs-CRP level of at least about 1 mg/L or at least about 2 mg/L prior to initiation of the treatment. In some embodiments, the subject has an hs-CRP level of at least about 1 mg/L prior to initiation of the treatment. In some embodiments, the treatment may be any of the dosing regimens described herein. In some embodiments, the subject has an hs-CRP level of at least about 2 mg/L prior to initiation of the treatment. In some embodiments, the subject has an hs-CRP level of at least about 3 mg/L prior to initiation of the treatment. In some embodiments, the subject is a pediatric patient and has an hs-CRP level of at least about 1 mg/L prior to initiation of the treatment. In some embodiments, the subject is an adolescent patient and has an hs-CRP level of at least about 1.5 mg/L prior to initiation of the treatment. In some embodiments, the subject is an adult patient and has an hs-CRP level of at least about 2 mg/L prior to initiation of the treatment.

In some embodiments, the hs-CRP level of the subject is reduced by at least 20%, at least 30%, or at least 40% after administration of the antibody or antigen binding fragment thereof. In some embodiments, the hs-CRP level of the subject is reduced by at least 20% after administration of the antibody or antigen binding fragment thereof. In some embodiments, the hs-CRP level of the subject is reduced by at least 30% after administration of the antibody or antigen binding fragment thereof. In some embodiments, the hs-CRP level of the subject is reduced by at least 40% after administration of the antibody or antigen binding fragment thereof. Such reductions may be seen e.g., after one month of administration, after two months of administration, after three months of administration, after four months of administration, or after five months of administration of the antibody or antigen binding fragment thereof.

In some embodiments, the hs-CRP level of the subject is reduced by at least 20%, at least 30%, or at least 40%. In some embodiments, the hs-CRP level of the subject is reduced by at least 20%, at least 30%, or at least 40%. In some embodiments, the hs-CRP level of the subject is reduced by at least 20%, at least 30%, or at least 40%. In some embodiments, the hs-CRP level of the subject is reduced by at least 20%, at least 30%, or at least 40% after 1 month of the treatment. In some embodiments, the hs-CRP level of the subject is reduced by at least 20%, at least 30%, or at least 40% after 2 months of the treatment. In some embodiments, the hs-CRP level of the subject is reduced by at least 20%, at least 30%, or at least 40% after 3 months of the treatment. In some embodiments, the hs-CRP level of the subject is reduced by at least 20%, at least 30%, or at least 40% after 4 months of the treatment. In some embodiments, the hs-CRP level of the subject is reduced by at least 20%, at least 30%, or at least 40% after 5 months of the treatment. In some embodiments, the hs-CRP level of the subject is reduced by at least 20%, at least 30%, or at least 40% after 6 months of the treatment.

In some embodiments, the hs-CRP level in the subject is reduced to below about 3 mg/L, to below about 2 mg/L, or to below about 1 mg/L after administration of the antibody or antigen binding fragment thereof. In some embodiments, the hs-CRP level in the subject is reduced to below about 3 mg/L after administration of the antibody or antigen binding fragment thereof. In some embodiments, the hs-CRP level in the subject is reduced to below about 2 mg/L after administration of the antibody or antigen binding fragment thereof. In some embodiments, the hs-CRP level in the subject is reduced to below about 1 mg/L after administration of the antibody or antigen binding fragment thereof.

In some embodiments, the hs-CRP level in the subject is reduced to below about 3 mg/L, to below about 2 mg/L, or to below about 1 mg/L after 3 months of the treatment. In some embodiments, the hs-CRP level in the subject is reduced to below about 3 mg/L, to below about 2 mg/L, or to below about 1 mg/L after 4 months of the treatment. In some embodiments, the hs-CRP level in the subject is reduced to below about 3 mg/L, to below about 2 mg/L, or to below about 1 mg/L after 5 months of the treatment. In some embodiments, the hs-CRP level in the subject is reduced to below about 3 mg/L, to below about 2 mg/L, or to below about 1 mg/L after 6 months of the treatment.

In some embodiments, the manifestation or complication associated with sickle cell disease is pain. In some embodiments, the pain is selected from vaso-occlusive pain events, vaso-occlusive pain crises, and bone pain. In some embodiments, the pain is chronic pain. In some embodiments, the pain is acute pain.

In one aspect, provided herein is a method of reducing pain associated with sickle cell disease in a subject in need thereof, the method comprising administering to the subject a therapeutically effective amount of an antibody or antigen binding fragment that specifically binds to IL-1 beta.

In some embodiments, the pain is reduced by at least 0.4 in a self-reported pain level scoring from 0 to 10, compared to the average score calculated 1-2 weeks prior to administration of the antibody or antigen binding fragment thereof. In some embodiments, the pain is reduced by at least 0.4 in a self-reported pain level scoring from 0 to 10, compared to the average score during the 1-2 weeks prior to the initiation of the treatment. In some embodiments, the pain is reduced by at least about 10%, at least about 15%, at least about 20%, at least about 25%, at least about 30%, at least about 35%, at least about 40%, at least about 50%, at least about 60%, at least about 70%, at least about 80%, or at least about 90% compared to the average score calculated 1-2 weeks prior to administration of the antibody or antigen binding fragment thereof. In some embodiments, the pain is reduced by at least about 10%, at least about 15%, at least about 20%, at least about 25%, at least about 30%, at least about 35%, at least about 40%, at least about 50%, at least about 60%, at least about 70%, at least about 80%, or at least about 90% compared to the average score calculated 1-2 weeks prior to initiation of the treatment. Such reductions may be seen e.g., after one month of administration, after two months of administration, after three months of administration, after four months of administration, or after five months of administration of the antibody or antigen binding fragment thereof.

In some embodiments, the manifestation or complication associated with sickle cell disease is selected from growth delay, stunted growth, low body mass index (BMI), and/or low body weight. In some embodiments, the subject in need thereof has an increase in lean body mass, an increase in lean muscle mass, an increase in body mass index (BMI), an increase in body weight, and/or a reversal of growth delay after administration of the antibody or antigen binding fragment that specifically binds to IL-1 beta.

In some embodiments, the subject has an increase in weight of at least about 5%, at least about 10%, at least about 15%, or at least about 20% in comparison to subject weight prior to administration of the antibody or antigen binding fragment thereof. In some embodiments, subject weight is increased at least about 5%, at least about 10%, at least about 15%, or at least about 20% in comparison to subject weight prior to administration of the antibody or antigen binding fragment thereof. In some embodiments, the subject has an increase in body mass index (BMI) of at least about 5%, at least about 10%, at least about 15%, or at least about 20% in comparison to subject BMI prior to administration of the antibody or antigen binding fragment thereof. In some embodiments, the subject body mass index (BMI) is increased at least about 5%, at least about 10%, at least about 15%, or at least about 20% in comparison to subject BMI prior to administration of the antibody or antigen binding fragment thereof.

In some embodiments, the subject has an increase in weight of up to about 5%, up to about 10%, up to about 15%, or up to about 20% in comparison to subject weight prior to administration of the antibody or antigen binding fragment thereof. In some embodiments, the subject has an increase in body mass index (BMI) of up to about 5%, up to about 10%, up to about 15%, or up to about 20% in comparison to subject BMI prior to administration of the antibody or antigen binding fragment thereof. In some embodiments, the increase in weight and/or the increase in BMI is measured about 12 weeks, about 24 weeks, about 7 months, about 8 months, about 9 months, about 10 months, about 11 months, about 12 months, about 13 months, or about 14 months after first administration of the antibody or antigen binding fragment thereof. In some embodiments, the increase in weight and/or the increase in BMI is measured at least about 12 weeks, at least about 24 weeks, at least about 7 months, at least about 8 months, at least about 9 months, at least about 10 months, at least about 11 months, at least about 12 months, at least about 13 months, or at least about 14 months after first administration of the antibody or antigen binding fragment thereof.

In some aspects, presented herein are methods of improving growth delay, stunted growth, low body mass index (BMI), and/or low body weight associated with sickle cell disease in a subject in need thereof, the method comprising administering to the subject a therapeutically effective amount of an antibody or antigen binding fragment that specifically binds to IL-1 beta. In some aspects, presented herein are methods of increasing lean body mass, increasing lean muscle mass, increasing body mass index (BMI), increasing body weight, and/or reversing growth delay in a subject in need thereof, wherein the subject has sickle cell disease, the method comprising administering to the subject a therapeutically effective amount of an antibody or antigen binding fragment that specifically binds to IL-1 beta.

In one embodiment, the manifestation or complication associated with sickle cell disease is a need for hospitalization. In one embodiment, the manifestation or complication associated with sickle cell disease is hospitalization. In some embodiments, the need for hospitalization or the hospitalization is reduced by either (i) annual frequency of hospitalization and/or (ii) duration of hospitalization measured by the number of days annually.

In some embodiments, the annual frequency of hospitalization is reduced by at least 50%, at least 60%, at least 70%, at least 80%, at least 90%, or by 100% compared to the frequency in the last 12 months prior to the initiation of the treatment. In some embodiments, the annual frequency of hospitalization is reduced by at least 50%, at least 60%, at least 70%, at least 80%, at least 90%, or by 100% compared to the frequency in the last 12 months prior to the administration of the antibody or antigen binding fragment thereof. In some embodiments, the duration of hospitalization is reduced by at least 50%, at least 60%, at least 70%, at least 80%, at least 90%, or by 100% compared to the frequency in the last 12 months prior to the initiation of the treatment. In some embodiments, the duration of hospitalization is reduced by at least 50%, at least 60%, at least 70%, at least 80%, at least 90%, or by 100% compared to the average duration of hospitalization in the last 12 months prior to the after administration of the antibody or antigen binding fragment thereof. In some embodiments, the duration of hospitalization measured by the number of days annually is reduced by at least 50%, at least 60%, at least 70%, at least 80%, at least 90%, or by 100% compared to the frequency in the last 12 months prior to the initiation of the treatment. In some embodiments, the duration of hospitalization measured by the number of days annually is reduced by at least 50%, at least 60%, at least 70%, at least 80%, at least 90%, or by 100% compared to the average duration of hospitalization in the last 12 months prior to the after administration of the antibody or antigen binding fragment thereof.

In some embodiments, the manifestation or complication associated with sickle cell disease is the use of one or more narcotic analgesic agents by the subject. In some embodiments, the annual use of one or more narcotic analgesic agents by the subject is reduced by at least about 20%, by at least about 30%, by at least about 40%, or by at least about 50% as compared to the use in the last 12 months prior to administration of the antibody or antigen binding fragment thereof. In some embodiments, the annual use of one or more narcotic analgesic agents by the subject is reduced by at least about 20%, by at least about 30%, by at least about 40%, or by at least about 50% as compared to the use in the last 12 months prior to initiation of the treatment.

In some embodiments, the manifestation or complication associated with sickle cell disease is acute and/or chronic blood transfusion therapy for treatment of anemia. In some embodiments, the total amount of transfused blood is reduced by at least about 20%, by at least about 30%, by at least about 40%, or by at least about 50% as compared to the amount of blood transfused in the last 12 months prior to administration of the antibody or antigen binding fragment thereof. In some embodiments, the total amount of transfused blood is reduced by at least about 20%, by at least about 30%, by at least about 40%, or by at least about 50% as compared to the amount of blood transfused in the last 12 months prior to initiation of the treatment.

In some embodiments, the manifestation or complication associated with sickle cell disease is organ damage. In some embodiments, the organ damage is end organ damage. In some embodiments, the organ damage is damage to one or more organs selected from: spleen, brain, eyes, lungs, liver, heart, kidneys, penis, joints, bones, and skin.

In one aspect, provided herein is a method of preventing organ damage associated with sickle cell disease in a subject in need thereof, the method comprising administering to the subject a therapeutically effective amount of an antibody or antigen binding fragment that specifically binds to IL-1 beta. In some embodiments, the manifestation or complication associated with sickle cell disease is organ damage. In some embodiments, the organ damage is end organ damage. In some embodiments, the organ damage is damage to one or more organs selected from: spleen, brain, eyes, lungs, liver, heart, kidneys, penis, joints, bones, and skin.

In some embodiments, the organ damage is sickle cell disease associated nephropathy. In some embodiments, the manifestation or complication associated with sickle cell disease is stroke. In some embodiments, the stroke is selected from the group consisting of an ischemic stroke or a hemorrhagic stroke.

In some aspects, provided herein are methods of preventing stroke associated with sickle cell disease in a subject in need thereof, the method comprising administering to the subject a therapeutically effective amount of an antibody or antigen binding fragment that specifically binds to IL-1 beta.

In some embodiments, the antibody or antigen binding fragment that specifically binds to IL-1 beta is administered intravenously (i.v.). In some embodiments, the antibody or antigen binding fragment that specifically binds to IL-1 beta is administered subcutaneously (s.c.). In some embodiments, the antibody or antigen binding fragment that specifically binds to IL-1 beta is canakinumab. In some embodiments, the antibody or antigen binding fragment that specifically binds to IL-1 beta is gevokizumab.

In some embodiments, the antibody or antigen binding fragment that specifically binds to IL-1 beta is administered to the patient at about 30 mg to 600 mg. In some embodiments, the antibody or antigen binding fragment that specifically binds to IL-1 beta is administered to the patient every two weeks, every three weeks, every four weeks, every six weeks, every eight weeks, every nine weeks, or every twelve weeks.

In some embodiments, about 150 mg to about 400 mg of canakinumab is administered to the patient every four weeks, every six weeks, every eight weeks, or every twelve weeks. In some embodiments, canakinumab is administered to the patient at about 150 mg every two weeks, every three weeks, every four weeks, every eight weeks, or every twelve weeks. In some embodiments, canakinumab is administered to the patient at about 200 mg every two weeks, every three weeks, every four weeks, every eight weeks, or every twelve weeks. In some embodiments, canakinumab is administered to the patient at about 250 mg every two weeks, every three weeks, every four weeks, every eight weeks, or every twelve weeks. In some embodiments, canakinumab is administered to the patient at about 300 mg every two weeks, every three weeks, every four weeks, every eight weeks, or every twelve weeks. In some embodiments, canakinumab is administered to the patient at about 400 mg every two weeks, every three weeks, every four weeks, every eight weeks, or every twelve weeks.

In some embodiments the subject is administered a therapeutically effective amount of an additional therapeutic agent. In some embodiments the additional therapeutic agent is selected from an antibody or antigen binding fragment thereof that specifically binds to p selectin, L-glutamine oral powder, and/or an agent that increases fetal hemoglobin. In some embodiments, the additional therapeutic agent is selected from hydroxyurea, an antibody or antigen binding fragment thereof that specifically binds to p selectin, L-glutamine oral powder, voxelotor, stem cells comprising a lentiviral vector which inserts a functioning version of the HBB gene, and combinations thereof. In some embodiments, the additional therapeutic agent is hydroxyurea. In some embodiments, the antibody or antigen binding fragment thereof that specifically binds to p selectin is crizanlizumab. In some embodiments, crizanlizumab is administered to the patient at about 2.5 mg/kg, about 5 mg/kg, or about 7.5 mg/kg. In some embodiments, the crizanlizumab is administered to the patient every four weeks, optionally wherein the first 2 doses are 2 weeks apart.

In some embodiments, the IL-1 beta binding antibody or antigen binding fragment thereof is administered to the subject in combination with crizanlizumab on the same day, optionally not more than 2 hours apart or not more than one hour apart. In some embodiments, the antibody or antigen binding fragment thereof that specifically binds to IL-1 beta is administered to the subject in a loading phase followed by a maintenance phase, wherein the subject receives a higher amount of the antibody during the loading phase than during the maintenance phase over the same given period of time. In some embodiments, the loading phase is at least 3 months. In some embodiments, the administration interval within the loading phase is the same as that within the maintenance phase. In some embodiments, the dose within the loading phase (loading dose) is at least twice the amount of the dose within the maintenance phase (maintenance dose). In some embodiments, the loading dose is the same as the maintenance dose. In some embodiments, the administration interval within the maintenance phase is twice or three times as long as the interval within the loading phase.

In one aspect, provided herein is an antibody or antigen binding fragment that specifically binds to IL-1 beta for use in treating one or more manifestations or complications of sickle cell disease, optionally wherein the manifestation or complication is selected from: intravascular inflammation, endothelial dysfunction, pain, fatigue, hospitalization, poor sleep quality, work absences, school absences, narcotic use, acute blood transfusion therapy given for disease severity, chronic blood transfusion therapy given for disease severity, acute chest syndrome, bone infarct, avascular necrosis, osteonecrosis, stroke, cognitive dysfunction, priapism, infarction of penis, a cardiovascular disorder, growth delay, stunted growth, low body mass index (BMI), low body weight, and organ damage.

In one aspect, provided herein is a use of an antibody or antigen binding fragment that specifically binds to IL-1 beta for the manufacture of a medicament of the treatment of one or more manifestations or complications of sickle cell disease, optionally wherein the manifestation or complication is selected from: intravascular inflammation, endothelial dysfunction, pain, fatigue, hospitalization, poor sleep quality, work absences, school absences, narcotic use, acute blood transfusion therapy given for disease severity, chronic blood transfusion therapy given for disease severity, acute chest syndrome, bone infarct, avascular necrosis, osteonecrosis, stroke, cognitive dysfunction, priapism, infarction of penis, a cardiovascular disorder, growth delay, stunted growth, low body mass index (BMI), low body weight, and organ damage.

Additional aspects and embodiments of the described methods of uses are inherently embodied throughout the specification.

### BRIEF DESCRIPTION OF THE DRAWINGS

FIG. 1 is a series of graphs demonstrating the percent change in baseline of hs-CRP, total leukocytes, neutrophils, and monocytes in canakinumab (line with circles; shown at bottom of each graph) and placebo (straight line; shown at top of each graph) treated patients over several weeks of treatment. Mean and standard error are shown. Baseline is derived from mean of screening and pre-dosing.

FIG. 2 is a set of graphs demonstrating MCA (medial cranial artery) flow velocity (cm/s) in canakinumab (ACZ885) and placebo treated patients over time. The change from baseline in MCA flow velocities (cm/s) is shown at the right for both groups.

FIG. 3 is a series of graphs demonstrating changes in average daily pain, average daily fatigue, sleep and school or work absences in canakinumab (line with solid circles) and placebo (line with open circles) treated patients over several weeks of treatment. Mean and standard error are shown.

FIG. 4 is a graph showing outcome data for patient weight in kg over time for both placebo treated and canakinumab (ACZ885) treated patients. Dotted lines are shown at 12 and 24 week timepoints.

FIG. 5 is a graph showing outcome data for patient body mass index (BMI) in kg/m² for both placebo treated and canakinumab (ACZ885) treated patients.

### GENERAL DEFINITIONS

In order that the present disclosure may be more readily understood, certain terms are first defined. Additional definitions are set forth throughout the detailed description as required.

Throughout the description and claims of this specification, the words “comprise” and “contain” and variations of the words, for example “comprising” and “comprises”, mean “including but not limited to”, and do not exclude other components, integers, or steps. Moreover the singular encompasses the plural unless the context otherwise requires: in particular, where the indefinite article is used, the specification is to be understood as contemplating plurality as well as singularity, unless the context requires otherwise.

As used herein, the term “sickle cell disease” or “SCD” refers to a group of inherited red blood cell disorders in which affected persons have an abnormal protein in their red blood cells. More common types of sickle cell disease include Hemoglobin SS, (HbSS; also called sickle cell anemia, is usually the most severe type of this disorder); Hemoglobin SC (HbSC; usually mild); Hemoglobin Sβ thalassemia (of which there are two types: “0” and “+”; HbS beta 0-thalassemia is usually more severe; HbS beta+− thalassemia is usually less severe). More rare types of sickle cell disease include Hemoglobin SD, Hemoglobin SE, and Hemoglobin SO. Manifestations and complications of sickle cell disease depend on the patient and severity of the disease. Early manifestations and complications may include painful swelling of the hands and feet; dark urine; symptoms of anemia such as fatigue and paleness; and jaundice. Over time, sickle cell disease can lead to additional manifestations and complications such as infections, delayed and/or stunted growth, and episodes of pain (e.g., vaso-occlusive pain events or vaso-occlusive pain crises). Younger children who have sickle cell disease may be pain-free or have reduced pain between crises. Adolescents and adults (including young adults) may suffer chronic, ongoing pain. Additional manifestations and complications that may be present in patients include, but are not limited to, fatigue, hospitalization, poor sleep quality, narcotic use, acute blood transfusion therapy given for disease severity, chronic blood transfusion therapy given for disease severity, acute chest syndrome, bone infarct, avascular necrosis, osteonecrosis, stroke (e.g., ischemic stroke or hemorrhagic stroke), priapism, infarction of penis, low body weight, growth delays, low body-mass index, slowed growth, and cardiovascular disorders. Over time, sickle cell disease may harm a patient's organs including the spleen, brain, eyes, lungs, liver, heart, kidneys, penis, joints, bones, and/or skin.

Canakinumab (international nonproprietary name (INN) number 8836) is described in WO02/16436, which is hereby incorporated by reference in its entirety for this purpose. Canakinumab is a fully human monoclonal anti-human interleukin 1 beta (IL-1β or IL-1beta) antibody of the IgG1/k isotype, being developed for the treatment of interleukin 1 beta driven inflammatory diseases. It binds to human interleukin 1 beta and blocks the interaction of the cytokine with its receptors. The antagonism of the IL-1β mediated inflammation using canakinumab in lowering high sensitivity C-reactive protein (hs-CRP) and other inflammatory marker levels has shown an acute phase response in patients with Cryopyrin-Associated Periodic Syndrome (CAPS) and rheumatoid arthritis. This evidence has been replicated in patients with type 2 diabetes mellitus (T2DM) using canakinumab and with other IL-1β antibody therapies in development, although in T2DM reduction in hs-CRP levels did not translate to increased efficaciousness over standard of care treatment. Herein is described the results of a study treating patients with sickle cell disease using canakinumab Administration of canakinumab to the subjects with sickle cell disease significantly reduces several manifestations and complications of this disease.

Gevokizumab (CAS number 1129435-60-4; also known as XOMA052) is described in WO2007/002261, which is hereby incorporated by reference in its entirety for this purpose. Gevokizumab is a humanized monoclonal anti-human interleukin 1 beta (IL-1β or IL-1beta) antibody of the IgG2 isotype. It binds to human interleukin 1 beta and blocks the interaction of the cytokine with its receptors.

Crizanlizumab (CAS number 1690318-25-2) is described in WO2008/69999, which is hereby incorporated by reference in its entirety for this purpose. Crizanlizumab is a humanized monoclonal anti-human P selectin antibody of the IgG2 isotype. It binds to human P selectin.

In various embodiments, the anti-interleukin 1 beta antibodies (e.g., canakinumab or gevokizumab) and antigen binding fragments thereof disclosed herein are capable of binding to interleukin 1 beta and blocking the interaction of the cytokine with its receptors. In some embodiments, the anti-interleukin 1 beta antibodies and antigen binding fragments thereof L-1β inhibitors include but not be limited to canakinumab or an antigen binding fragment thereof, gevokizumab or an antigen binding fragment thereof, an IL-1 Affibody (SOBI 006, Z-FC (Swedish Orphan Biovitrum/Affibody)), CDP-484 (Celltech), and LY-2189102 (Lilly).

The term “antibody” as used herein refers to a whole antibody or antigen binding fragment thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes, but is not limited to, monoclonal antibodies, human antibodies, humanized antibodies, camelised antibodies, and chimeric antibodies. The antibodies can be of any isotype/class (e.g., IgG, IgE, IgM, IgD, IgA and IgY) or subclass (e.g., IgG₁, IgG₂, IgG₃, IgG₄, IgA₁, and IgA₂).

The term “antigen binding fragment” refers to a fragment of an intact antibody that retains the ability to specifically bind to a given antigen (e.g., interleukin 1 beta) and/or provide a function of the intact antibody. Such fragments include Fab fragments, Fab′ fragments, monovalent fragments consisting of the VL, VH, CL and CH1 domains; F(ab′)2 fragments, bivalent fragments comprising two Fab fragments linked by a disulfide bridge at the hinge region; an Fd fragment consisting of the VH and CH1 domains; an Fv fragment consisting of the VL and VH domains, a single chain Fv fragment (scFv) consisting of the VL and VH domains connected by a linker sequence; and a single domain antibody (dAb) fragment (Ward et al., 1989 Nature 341:544-546), which consists of a VH domain or a VL domain.

The term “single chain antibody”, “single chain Fv” or “scFv” is refers to a molecule comprising an antibody heavy chain variable domain (or region; VH) and an antibody light chain variable domain (or region; VL) connected by a linker. Such scFv molecules can have the general structures: NH2-VL-linker-VH-COOH or NH2-VH-linker-VL-COOH. Any suitable linker may be used. A non-limiting set of linkers that can be used in such single chain antibodies are described by Holliger et al. (1993), Proc. Natl. Acad. Sci. USA 90:6444-6448, Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol; the contents of each of which are herein incorporated by reference for this purpose. Such single chain antibodies are also intended to be encompassed within the term “antigen binding fragment” of an antibody. These antibody fragments are obtained using techniques known to those of skill in the art, and the fragments are screened for utility in the same manner as are intact antibodies. Without limitation, an antigen binding fragment can be produced by any suitable method known in the art. For instance, the various antigen binding fragments described herein can be produced by enzymatic or chemical modification of intact antibodies, synthesized de novo using recombinant DNA methodologies (e.g., single chain Fv), or identified using phage display libraries (see, e.g., Pini and Bracci, Curr Protein Pept Sci 2000; 1(2):155-69, the contents of which are herein incorporated by reference for this purpose). Antigen binding fragments are screened for utility (e.g., binding affinity, activity) in the same manner as are intact antibodies.

Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136, the contents of which are herein incorporated by reference for this purpose). Antigen binding portions of antibodies can be grafted into scaffolds based on polypeptides such as Fibronectin type III (Fn3) (see e.g., U.S. Pat. No. 6,703,199, which describes fibronectin polypeptide monobodies, the contents of which are herein incorporated by reference for this purpose).

Antigen binding fragments can be incorporated into single chain molecules comprising a pair of tandem Fv segments (VH-CH1-VH-CH1) which, together with complementary light chain polypeptides, form a pair of antigen binding regions (see Zapata et al., 1995 Protein Eng. 8(10):1057-1062; and U.S. Pat. No. 5,641,870; the contents of each of which are herein incorporated by reference for this purpose).

Throughout this specification, complementarity determining regions (“CDR”) are defined according to the Kabat definition unless specified that the CDR are defined according to another definition. The precise amino acid sequence boundaries of a given CDR can be determined using any of a number of well-known schemes, including those described by Kabat et al. (1991), “Sequences of Proteins of Immunological Interest,” 5^{th} Ed. Public Health Service, National Institutes of Health, Bethesda, Md. (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme); the contents of each of which are herein incorporated by reference for this purpose. For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

By convention, the CDR regions in the heavy chain are typically referred to as HCDR1, HCDR2 and HCDR3 and in the light chain as LCDR1, LCDR2 and LCDR3. They are numbered sequentially in the direction from the amino terminus to the carboxy terminus.

The term “antibody framework” as used herein refers to the part of the variable domain, either VL or VH, which serves as a scaffold for the antigen binding loops (CDRs) of this variable domain. In essence, it is the variable domain without the CDRs.

The terms “constant region” or “constant domain” refer to a carboxy terminal portion of the light and heavy chain which is not directly involved in binding of the antibody to antigen but exhibits various effector functions, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

Binding “affinity” refers to the strength of interaction between antibody and antigen at single antigenic sites. Within each antigenic site, the variable region of the antibody “arm” interacts through weak non-covalent forces with antigen at numerous sites. In general, the more interactions, the stronger the affinity. Generally, such determinations can be made using a cell-based assay.

The term “Kassoc” or “Ka”, as used herein, is intended to refer to the association rate of a particular binding molecule-antigen interaction, whereas the term “Kdis” or “Kd,” as used herein, is intended to refer to the dissociation rate of a particular binding molecule-antigen interaction. The term “K_{D}”, as used herein, is intended to refer to the equilibrium dissociation constant, which is obtained from the ratio of Kd to Ka (i.e., Kd/Ka) and is expressed as a molar concentration (M). K_{D} values for antibodies can be determined using methods well established in the art. A method for determining the K_{D} of an antibody is by using surface plasmon resonance, such as a Biacore® system, or solution equilibrium titration (SET) (see Friguet et al., (1985) J. Immunol. Methods, 77(2):305-319, and Hanel et al., (2005) Anal. Biochem., 339(1):182-184), the contents of each of which are herein incorporated by reference for this purpose.

As used herein, the term “specific,” “specifically binds,” and “binds specifically” refers to a binding reaction between an antibody or antigen binding fragment (e.g., an anti-interleukin 1 beta antibody) and a target antigen (e.g., interleukin 1 beta) in a heterogeneous population of proteins and other biologics. Antibodies can be tested for specificity of binding by comparing binding to an appropriate antigen to binding to an irrelevant antigen or antigen mixture under a given set of conditions. If the antibody binds to the appropriate antigen with at least 2, 5, 7, and preferably 10 or more times more affinity than to the irrelevant antigen or antigen mixture, then it is considered to be specific. A “specific antibody” or a “target-specific antibody” is one that only binds the target antigen (e.g., interleukin 1 beta), but does not bind (or exhibits minimal binding) to other antigens. In certain embodiments, an antibody or antigen binding fragment that specifically binds the target antigen (e.g., interleukin 1 beta) has a K_{D} of less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹²M, or less than 1×10⁻¹³ M. In certain embodiments, the K_{D} is about 1 pM to about 600 pM. In certain embodiments, the K_{D} is between 600 pM to 1 μM, 1 μM to 100 nM, or 100 mM to 10 nM (inclusive). In some embodiments, an antibody that specifically binds to interleukin 1 beta may be gevokizumab or canakinumab. In some embodiments, and antibody that specifically binds to p selectin may be crizanlizumab.

Unless otherwise specifically stated or clear from context, as used herein, the term “about” in relation to a numerical value is understood as being within the normal tolerance in the art, e.g., within two standard deviations of the mean. Thus, “about” can be within +1-10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

### Uses and Methods of Treatment

Methods of Treatment

Provided herein are methods of treating, preventing, reducing, or eliminating a manifestation or complication associated with sickle cell disease in a subject in need thereof, optionally wherein the manifestation or complication is selected from: pain, fatigue, hospitalization, poor sleep quality, narcotic use, acute blood transfusion therapy given for disease severity, chronic blood transfusion therapy given for disease severity, acute chest syndrome, bone infarct, avascular necrosis, osteonecrosis, stroke, priapism, infarction of penis, a cardiovascular disorder, growth delay, stunted growth, low body mass index (BMI), low body weight, organ damage (e.g., end organ damage), cognitive dysfunction, intravascular inflammation, and endothelial dysfunction. In some embodiments, the methods of treating, preventing, reducing, or eliminating a manifestation or complication associated with sickle cell disease in a subject in need thereof will result in improved daily quality of life (including, e.g., self-reported daily quality of life and/or observed quality of life).

It shall be understood that the effects demonstrated herein for treating, preventing, reducing, or eliminating a manifestation or complication associated with sickle cell disease in a subject in need thereof may be demonstrated after a period of administering a treatment regimen described herein. For example, in one embodiment, the treatment, reduction, or elimination of a manifestation or complication of sickle cell disease after three months may be achieved by the administration of the antibody or antigen binding fragment thereof that specifically binds to IL-1 beta (e.g., canakinumab) at a certain dose and/or a certain administration schedule (e.g., about 200 mg administered every two, three or four weeks; about 250 mg administered every two, three, or four weeks; or 300 mg administered every two, three, or four weeks), optionally in combination with an additional therapeutic agent as described herein (e.g., crizanlizumab and/or hydroxyurea).

The term “administering” in relation to a compound, e.g., an anti-interleukin 1 beta antibody or another agent, is used to refer to delivery of that compound to a patient by any route.

As used herein, the term “patient” and “subject” are interchangeable.

In some embodiments, treatment of the manifestations or complications of sickle cell disease may comprise preventing or reducing severity and/or duration of the one or more manifestation(s) or complication(s) of sickle cell disease, wherein each of the manifestation(s) or complication(s) may be selected from: pain, fatigue, hospitalization, poor sleep quality, narcotic use, acute blood transfusion therapy given for disease severity, chronic blood transfusion therapy given for disease severity, acute chest syndrome, bone infarct, avascular necrosis, osteonecrosis, stroke, cognitive dysfunction, priapism, infarction of penis, a cardiovascular disorder, growth delay, stunted growth, low body mass index (BMI), low body weight, and organ damage.

In some embodiments, the subject may be a pediatric patient or pediatric subject. In some embodiments, the pediatric patient is under 18 years old. In some embodiments the pediatric patient may be an adolescent of between 12 and 18 years. In some embodiments, the pediatric patient may be a child of under 12 years. In some embodiments, the subject may be an adult patient. In some embodiments, the adult patient is 18 years old or older. In some embodiments, the adult patient is 21 years old or older.

In some embodiments the cardiovascular disorder is any cardiovascular disorder associated with sickle cell disease. In certain embodiments, the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI). In certain embodiments, the heart failure is selected from a heart failure with reduced ejection fraction (HFrEF), heart failure with preserved ejection fraction (HFpEF), heart failure after acute myocardial infarct, or acute decompensated heart failure. In certain embodiments, the hypertrophic cardiomyopathy is ventricular hypertrophy. In certain embodiments, the hypertension is selected from resistant hypertension, hypertensive heart disease, pulmonary hypertension, pulmonary arterial hypertension, isolated systolic hypertension, resistant hypertension, and pulmonary arterial hypertension. In one embodiment, the hypertension is sickle cell disease associated pulmonary hypertension. In some embodiments, canakinumab may be administered to a subject with sickle cell disease to prevent pulmonary hypertension.

In some embodiments, the organ damage is damage to one or more organs selected from: spleen, brain, eyes, lungs, liver, heart, kidneys, penis, joints, bones, and skin. Such organ damage may be any type known to be caused by sickle cell disease. In some embodiments, the organ damage is end organ damage. In some embodiments, the organ damage is kidney damage. In some embodiments, the kidney damage or damage to the kidneys is sickle cell nephropathy. In some embodiments, one kidney is damaged. In some embodiments, both kidneys are damaged. In some embodiments, the organ damage is eye damage. In some embodiments, one eye is damaged. In some embodiments, both eyes are damaged. In some embodiments, the organ damage is damage to the spleen. In some embodiments, the damage is to the brain. In some embodiments the organ damage is stroke (e.g., ischemic stroke or a hemorrhagic stroke).

In some embodiments, canakinumab may be administered to a subject with sickle cell disease to treat or prevent kidney damage (e.g., sickle cell nephropathy) in one or both kidneys. In some embodiments, canakinumab may be administered to a subject with sickle cell disease to treat kidney damage (e.g., sickle cell nephropathy) in one or both kidneys. In some embodiments, canakinumab may be administered to a subject with sickle cell disease who suffers from kidney damage to prevent further kidney damage (e.g., sickle cell nephropathy) in one or both kidneys. In some embodiments, canakinumab may be administered to a subject with sickle cell disease to prevent acute papillary necrosis in one or both kidneys. In some embodiments, canakinumab may be administered to a subject with sickle cell disease to prevent acute kidney damage in one or both kidneys. In some embodiments, canakinumab may be administered to a subject with sickle cell disease to prevent chronic kidney damage in one or both kidneys. In some embodiments, canakinumab may be administered to a subject with sickle cell disease to prevent progression to end-stage renal failure.

In some embodiments, canakinumab may be administered to a subject with sickle cell disease to prevent stroke (e.g., ischemic stroke or a hemorrhagic stroke). In some embodiments, canakinumab may be administered to a subject with sickle cell disease who has already had one or more strokes to prevent an additional stroke (e.g., ischemic stroke or a hemorrhagic stroke).

In some embodiments, canakinumab may be administered to a subject with sickle cell disease to treat, prevent, or reduce intravascular inflammation. In some embodiments, canakinumab may be administered to a subject with sickle cell disease to treat, prevent, or reduce endothelial dysfunction.

In some embodiments, the organ damage is eye damage. In some embodiments, the damage is to one eye. In some embodiments, the damage is to both eyes. In some embodiments, the damage to the eye or eyes is deterioration of vision (i.e., results in a an increased prescription). In some embodiments, the damage to the eye or eyes is loss of vision (i.e., total blindness or legal blindness in more than one eye). In some embodiments, the damage to the eye or eyes is retinopathy (e.g., background or proliferative). In some embodiments, the damage to the eye or eyes is vitreous hemorrhage. In some embodiments, the damage to the eye or eyes is loss of vision (i.e., total blindness or legal blindness in more than one eye). In some embodiments, the damage to the eye or eyes is retinal detachment. In some embodiments, canakinumab may be administered to a subject with sickle cell disease to prevent or reduce deterioration of vision (i.e., damage resulting in an increased prescription). In some embodiments, canakinumab may be administered to a subject with sickle cell disease to prevent or reduce loss of vision (i.e., total blindness or legal blindness in one eye or both eyes). In some embodiments, canakinumab may be administered to a subject with sickle cell disease to prevent or reduce retinopathy (e.g., background or proliferative). In some embodiments, canakinumab may be administered to a subject with sickle cell disease to prevent or reduce vitreous hemorrhage. In some embodiments, canakinumab may be administered to a subject with sickle cell disease to prevent or reduce loss of vision (i.e., total blindness or legal blindness in one eye or both eyes). In some embodiments, canakinumab may be administered to a subject with sickle cell disease to prevent or reduce retinal detachment.

In some embodiments, canakinumab may be administered to a subject with sickle cell disease to prevent splenic infarction and subsequent hyposplenism or functional asplenism. In some embodiments, canakinumab may be administered to a subject with sickle cell disease to prevent splenic infarction. In some embodiments, canakinumab may be administered to a subject with sickle cell disease to prevent hyposplenism. In some embodiments, canakinumab may be administered to a subject with sickle cell disease to prevent functional asplenism. In some embodiments, canakinumab may be administered to a subject with sickle cell disease to reduce susceptibility to infection (e.g., due to functional aspenism).

In some embodiments, canakinumab may be administered to a subject with sickle cell disease to prevent osteonecrosis. In some embodiments, canakinumab may be administered to a subject with sickle cell disease to prevent bone infarct. In some embodiments, canakinumab may be administered to a subject with sickle cell disease to prevent or reduce the need for joint replacement (e.g., due to osteonecrosis and/or bone infarct).

In some embodiments, the subject has any type of sickle cell disease described herein. In some embodiments, the subject has HbSS thalassemia. In some embodiments, the subject has HbS-beta⁰ thalassemia.

In some embodiments, the subject has reduced daily pain. In some embodiments, the subject has reduced daily pain levels. In some embodiments, the subject has increased time free from pain (e.g., increased minutes, hours, days, or weeks free from pain). In some embodiments, the pain is vaso-occlusive pain events or vaso-occlusive pain crises. In some embodiments, the pain is bone pain.

In some embodiments, the manifestation or complication associated with sickle cell disease is selected from growth delay, stunted growth, low body mass index (BMI), and/or low body weight. In some embodiments, the subject suffers from growth delay, stunted growth, low body mass index (BMI), and/or low body weight. In some embodiments, the subject has an increase in lean body mass, an increase in lean muscle mass, an increase in body mass index (BMI), an increase in body weight, and/or a reversal of growth delay after administration of the antibody or antigen binding fragment that specifically binds to IL-1 beta (e.g., canakinumab) In some embodiments, the subject has an increase in lean body mass after administration of the antibody or antigen binding fragment that specifically binds to IL-1 beta (e.g., canakinumab) In some embodiments, the subject has an increase in lean muscle mass after administration of the antibody or antigen binding fragment that specifically binds to IL-1 beta (e.g., canakinumab) In some embodiments, the subject has an increase in body mass index (BMI) after administration of the antibody or antigen binding fragment that specifically binds to IL-1 beta (e.g., canakinumab) In some embodiments, the subject has an increase in body weight after administration of the antibody or antigen binding fragment that specifically binds to IL-1 beta (e.g., canakinumab) In some embodiments, the subject has a reversal of growth delay after administration of the antibody or antigen binding fragment that specifically binds to IL-1 beta (e.g., canakinumab).

In some embodiments, the subject has an increase in weight of at least about 5%, at least about 10%, at least about 15%, or at least about 20% in comparison to subject weight prior to administration of the antibody or antigen binding fragment thereof (e.g., canakinumab) In some embodiments, the subject has an increase in weight of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% in comparison to subject weight prior to administration of the antibody or antigen binding fragment thereof (e.g., canakinumab) In some embodiments, the subject has an increase in body mass index (BMI) of at least about 5%, at least about 10%, at least about 15%, or at least about 20% in comparison to subject BMI prior to administration of the antibody or antigen binding fragment thereof (e.g., canakinumab) In some embodiments, the subject has an increase in body mass index (BMI) of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% in comparison to subject BMI prior to administration of the antibody or antigen binding fragment thereof (e.g., canakinumab).

In some embodiments, the subject has an increase in weight of up to about 5%, up to about 10%, up to about 15%, or up to about 20% in comparison to subject weight prior to administration of the antibody or antigen binding fragment thereof (e.g., canakinumab) In some embodiments, the subject has an increase in weight of up to about 5%, up to about 6%, up to about 7%, up to about 8%, up to about 9%, up to about 10%, up to about 11%, up to about 12%, up to about 13%, up to about 14%, up to about 15%, up to about 16%, up to about 17%, up to about 18%, up to about 19%, or up to about 20% in comparison to subject weight prior to administration of the antibody or antigen binding fragment thereof (e.g., canakinumab).

In some embodiments, the subject has an increase in body mass index (BMI) of up to about 5%, up to about 10%, up to about 15%, or up to about 20% in comparison to subject BMI prior to administration of the antibody or antigen binding fragment thereof (e.g., canakinumab) In some embodiments, the subject has an increase in body mass index (BMI) of up to about 5%, up to about 6%, up to about 7%, up to about 8%, up to about 9%, up to about 10%, up to about 11%, up to about 12%, up to about 13%, up to about 14%, up to about 15%, up to about 16%, up to about 17%, up to about 18%, up to about 19%, or up to about 20% in comparison to subject BMI prior to administration of the antibody or antigen binding fragment thereof (e.g., canakinumab).

In some embodiments, the subject has an increase in weight of at least about 5%, at least about 10%, at least about 15%, or at least about 20% after any of the treatments described herein in comparison to prior to the treatment. In some embodiments, the subject has an increase in weight of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% after any of the treatments described herein in comparison to prior to the treatment. In some embodiments, the subject has an increase in body mass index (BMI) of at least about 5%, at least about 10%, at least about 15%, or at least about 20% after any of the treatments described herein in comparison to prior to the treatment. In some embodiments, the subject has an increase in body mass index (BMI) of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% after any of the treatments described herein in comparison to prior to the treatment.

In some embodiments, the subject has an increase in weight of up to about 5%, up to about 10%, up to about 15%, or up to about 20% after any of the treatments described herein in comparison to prior to the treatment. In some embodiments, the subject has an increase in weight of up to about 5%, up to about 6%, up to about 7%, up to about 8%, up to about 9%, up to about 10%, up to about 11%, up to about 12%, up to about 13%, up to about 14%, up to about 15%, up to about 16%, up to about 17%, up to about 18%, up to about 19%, or up to about 20% after any of the treatments described herein in comparison to prior to the treatment.

In some embodiments, the subject has an increase in body mass index (BMI) of up to about 5%, up to about 10%, up to about 15%, or up to about 20% after any of the treatments described herein in comparison to prior to the treatment. In some embodiments, the subject has an increase in body mass index (BMI) of up to about 5%, up to about 6%, up to about 7%, up to about 8%, up to about 9%, up to about 10%, up to about 11%, up to about 12%, up to about 13%, up to about 14%, up to about 15%, up to about 16%, up to about 17%, up to about 18%, up to about 19%, or up to about 20% after any of the treatments described herein in comparison to prior to the treatment.

In some embodiments, the increase in weight and/or the increase in BMI is measured about 12 weeks, about 24 weeks, about 7 months, about 8 months, about 9 months, about 10 months, about 11 months, about 12 months, about 13 months, or about 14 months after first administration of the antibody or antigen binding fragment thereof (e.g., canakinumab) In some embodiments, the increase in weight and/or the increase in BMI is measured at least about 12 weeks, at least about 24 weeks, at least about 7 months, at least about 8 months, at least about 9 months, at least about 10 months, at least about 11 months, at least about 12 months, at least about 13 months, or at least about 14 months after first administration of the antibody or antigen binding fragment thereof (e.g., canakinumab).

In some aspects or embodiments, provided herein is an antibody or antigen binding fragment thereof that specifically binds to IL-1 beta (e.g., canakinumab) for use in improving growth delay, stunted growth, low body mass index (BMI), and/or low body weight associated with sickle cell disease in a subject in need thereof, the method comprising administering to the subject a therapeutically effective amount of an antibody or antigen binding fragment that specifically binds to IL-1 beta (e.g., canakinumab) In some aspects or embodiments, provided herein is an antibody or antigen binding fragment thereof that specifically binds to IL-1 beta (e.g., canakinumab) for use in increasing lean body mass, increasing lean muscle mass, increasing body mass index (BMI), increasing body weight, and/or reversing growth delay in a subject in need thereof, wherein the subject has sickle cell disease, the method comprising administering to the subject a therapeutically effective amount of an antibody or antigen binding fragment that specifically binds to IL-1 beta (e.g., canakinumab).

In some embodiments, canakinumab may be administered to a subject with sickle cell disease to prevent growth delay. In some embodiments, canakinumab may be administered to a subject with sickle cell disease to treat growth delay. In some embodiments, canakinumab may be administered to a subject with sickle cell disease to increase lean body mass. In some embodiments, canakinumab may be administered to a subject with sickle cell disease to increasing body mass index (BMI). In some embodiments, canakinumab may be administered to a subject with sickle cell disease to reverse growth delay. In some embodiments, canakinumab may be administered to a subject with sickle cell disease to increase body weight.

In some embodiments, canakinumab may be administered to a subject in need thereof to treat growth delay associated with sickle cell disease. In some embodiments, canakinumab may be administered to a subject in need thereof to treat stunted growth associated with sickle cell disease. In some embodiments, canakinumab may be administered to a subject in need thereof to treat low body mass index (BMI) associated with sickle cell disease. In some embodiments, canakinumab may be administered to a subject in need thereof to treat low body weight associated with sickle cell disease.

In some embodiments, the subject has a body weight (e.g., in kg) or a body mass index (BMI; e.g., in kg/m²) closer to that of peers or siblings who do not have sickle cell disease or sickle cell anemia.

In some embodiments, the subject has reduced fatigue. In some embodiments, the subject reports feeling more energetic. In some embodiments, the subject reports feeling less fatigued overall. In some embodiments, the subject has a reduced annular rate of hospitalization. In some embodiments, after administration of the treatment, the subject spends less days in the hospital per year. In some embodiments, after administration of the treatment, the subject spends overall time (days, weeks, or months) in the hospital. In some embodiments, after administration of the treatment, the subject has reduced numbers of visits to the hospital. In some embodiments, after administration of the treatment, the subject has reduced duration of visits to the hospital. In some embodiments, after administration of the treatment, the subject has reduced number and duration of visits to the hospital.

In some embodiments, the subject has improved sleep quality. In some embodiments, after administration of the treatment, the subject has increased duration of sleep. In some embodiments, after administration of the treatment, the subject has decreased waking events. In some embodiments, after administration of the treatment, the subject has less fragmented sleep. In some embodiments, the less fragmented sleep is calculated using any means known in the field. In some embodiments, the less fragmented sleep is calculated using means described herein. In some embodiments, after administration of the treatment, the subject has improved nocturnal sleep quality.

In some embodiments, the subject has decreased work absences. In some embodiments, the subject has a decreased number of work absences. In some embodiments, the subject has decreased duration of work absences. In some embodiments the subject has a decrease in the number and duration of work absences.

In some embodiments, the subject has decreased school absences. In some embodiments, the subject has a decreased number of school absences. In some embodiments, the subject has decreased duration of school absences. In some embodiments the subject has a decrease in the number and duration of school absences.

In some embodiments, after administration of the treatment, the subject has reduced use of one or more narcotic analgesic agents. In some embodiments, the narcotic use may be use of any narcotic agent, e.g., one or more of the following narcotic analgesic agents: morphine, fentanyl, oxymorphone, oxycodone, hydromorphone, buprenorphine, meperidine, methadone, opium, levorphanol, tramadol, nalbuphine, propoxyphene, tapentadol, or pentazocine.

In some embodiments, the subject has reduced intravascular inflammation and/or endothelial dysfunction associated with sickle cell disease after administration of the treatment. In some embodiments, this reduction may be demonstrated by reduction of serum inflammatory biomarkers and/or transcranial Doppler velocities.

In one embodiment the intravascular inflammation is reduced, whereby hs-CRP in the subject is reduced by at least 20%, at least 30%, at least 40% as compared to the level just prior to the initiation of any of the treatments described herein. In one embodiment the intravascular inflammation is reduced, whereby hs-CRP in the subject is reduced by at least 20%, at least 30%, or at least 40% as compared to the level just prior to the initiation of any of the treatments described herein at 3 months after any of the treatments described herein.

In one embodiment the intravascular inflammation is reduced, whereby leutocytes in the subject are reduced by at least 10%, or at least 15% as compared to the level just prior to the initiation of any of the treatments described herein. In one embodiment the intravascular inflammation is reduced, whereby leutocytes in the subject is reduced by at least 10%, at least 15% as compared to the level just prior to the initiation of any of the treatments described herein at 3 months after any of the treatments described herein.

In one embodiment the intravascular inflammation is reduced, whereby neutrophils in the subject are reduced by at least 15%, at least 20% as compared to the level just prior to the initiation of any of the treatments described herein. In one embodiment the intravascular inflammation is reduced, whereby neutrophils in the subject is reduced by at least 15%, at least 20% as compared to the level just prior to the initiation of any of the treatments described herein at 3 months after any of the treatments described herein.

In one embodiment the intravascular inflammation is reduced, whereby monocytes in the subject are reduced by at least 5%, at least 10% as compared to the level just prior to the initiation of any of the treatments described herein. In one embodiment the intravascular inflammation is reduced, whereby monocytes in the subject is reduced by at least 5%, at least 10% as compared to the level just prior to the initiation of any of the treatments described herein at 3 months after any of the treatments described herein.

In some embodiments, the subject has reduced cognitive dysfunction (i.e., has improved cognitive function) or does not have an increase in cognitive dysfunction associated with sickle cell disease after administration of the treatment. Cognitive dysfunction (CD) as used herein may be defined as significant deficits in any or all of the following main cognitive functions: memory (learning and recall), complex attention, simple attention, executive skills (planning, organizing, and sequencing), visual-spatial processing, language (e.g. verbal, fluency), reasoning/problem solving and psychomotor speed. Certain neuropsychological tests may be used to monitor or determine the levels of cognitive dysfunction in a patient including those used to determine any of the above parameters such as psychomotor speed, attention, memory (episodic memory, spatial working memory (working memory and executive function) and attention span (e.g., sustained attention). Reduction in cognitive dysfunction may be associated with improvement or lack of reduction in any one of the described cognitive functions. Additionally, patient reported and/or guardian reported improvements in cognitive function or maintenance of cognitive function after administration of the treatment may provide evidence that the treatment has been successful. In some embodiments, canakinumab may be administered to a subject with sickle cell disease to prevent or reduce memory loss. In some embodiments, canakinumab may be administered to a subject with sickle cell disease to prevent or reduce cognitive dysfunction. In some embodiments, canakinumab may be administered to a subject with sickle cell disease to improve cognitive function. In some embodiments, canakinumab may be administered to a subject with sickle cell disease to improve school performance. In some embodiments, canakinumab may be administered to a subject with sickle cell disease to improve concentration time.

As a set of non-limiting examples, the anti-interleukin 1 beta antibody (e.g., canakinumab or gevokizumab) may be administered to a subject with sickle cell disease at about 100 mg to about 200 mg, about 200 mg to about 400 mg, or about 400 mg to about 600 mg. As a set of non-limiting examples, the anti-interleukin 1 beta antibody (e.g., canakinumab or gevokizumab) may be administered to a subject with sickle cell disease at about 200 mg to about 300 mg, about 300 mg to about 400 mg, about 400 mg to about 500 mg, or about 500 mg to about 600 mg. As a set of non-limiting examples, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, or about 500 mg to about 600 mg. As a set of non-limiting examples, the anti-interleukin 1 beta antibody (e.g., canakinumab or gevokizumab) may be administered to a subject with sickle cell disease at about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg. Such administrations of the anti-interleukin 1 beta antibody (e.g., canakinumab or gevokizumab) to the subject with sickle cell disease may occur, e.g., every two weeks, every three weeks, every four weeks, every five weeks, every six weeks, every seven weeks, every eight weeks, every nine weeks, every ten weeks, every eleven weeks, or every twelve weeks. Such administrations would, for example, reduce the severity and/or duration of one or more manifestation(s) or complication(s) of sickle cell disease, wherein each of the manifestation(s) or complication(s) may be selected from any complication of sickle cell disease including, but not limited to, pain, fatigue, hospitalization, poor sleep quality, work absences, school absences, narcotic use, acute blood transfusion therapy given for disease severity, chronic blood transfusion therapy given for disease severity, acute chest syndrome, bone infarct, avascular necrosis, osteonecrosis, stroke, cognitive dysfunction, priapism, infarction of penis, a cardiovascular disorder, growth delay, stunted growth, low body mass index (BMI), low body weight, and organ damage.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 100 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 100 mg once every two weeks. In some embodiments, the antibody or antigen binding fragment that specifically binds to IL-1 beta (e.g., canakinumab or gevokizumab) is administered to the subject at about 100 mg once every three weeks. In some embodiments, the antibody or antigen binding fragment that specifically binds to IL-1 beta (e.g., canakinumab or gevokizumab) is administered to the subject at about 100 mg once every four weeks. In some embodiments, the antibody or antigen binding fragment that specifically binds to IL-1 beta (e.g., canakinumab or gevokizumab) is administered to the subject at about 100 mg once every five weeks. In some embodiments, the antibody or antigen binding fragment that specifically binds to IL-1 beta (e.g., canakinumab or gevokizumab) is administered to the subject at about 100 mg once every six weeks. In some embodiments, the antibody or antigen binding fragment that specifically binds to IL-1 beta (e.g., canakinumab or gevokizumab) is administered to the subject at about 100 mg once every seven weeks. In some embodiments, the antibody or antigen binding fragment that specifically binds to IL-1 beta (e.g., canakinumab or gevokizumab) is administered to the subject at about 100 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 125 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 125 mg once every two weeks. In some embodiments, the antibody or antigen binding fragment that specifically binds to IL-1 beta (e.g., canakinumab or gevokizumab) is administered to the subject at about 125 mg once every three weeks. In some embodiments, the antibody or antigen binding fragment that specifically binds to IL-1 beta (e.g., canakinumab or gevokizumab) is administered to the subject at about 125 mg once every four weeks. In some embodiments, the antibody or antigen binding fragment that specifically binds to IL-1 beta (e.g., canakinumab or gevokizumab) is administered to the subject at about 125 mg once every five weeks. In some embodiments, the antibody or antigen binding fragment that specifically binds to IL-1 beta (e.g., canakinumab or gevokizumab) is administered to the subject at about 125 mg once every six weeks. In some embodiments, the antibody or antigen binding fragment that specifically binds to IL-1 beta (e.g., canakinumab or gevokizumab) is administered to the subject at about 125 mg once every seven weeks. In some embodiments, the antibody or antigen binding fragment that specifically binds to IL-1 beta (e.g., canakinumab or gevokizumab) is administered to the subject at about 125 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 150 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 150 mg once every two weeks. In some embodiments, the antibody or antigen binding fragment that specifically binds to IL-1 beta (e.g., canakinumab or gevokizumab) is administered to the subject at about 150 mg once every three weeks. In some embodiments, the antibody or antigen binding fragment that specifically binds to IL-1 beta (e.g., canakinumab or gevokizumab) is administered to the subject at about 150 mg once every four weeks. In some embodiments, the antibody or antigen binding fragment that specifically binds to IL-1 beta (e.g., canakinumab or gevokizumab) is administered to the subject at about 150 mg once every five weeks. In some embodiments, the antibody or antigen binding fragment that specifically binds to IL-1 beta (e.g., canakinumab or gevokizumab) is administered to the subject at about 150 mg once every six weeks. In some embodiments, the antibody or antigen binding fragment that specifically binds to IL-1 beta (e.g., canakinumab or gevokizumab) is administered to the subject at about 150 mg once every seven weeks. In some embodiments, the antibody or antigen binding fragment that specifically binds to IL-1 beta (e.g., canakinumab or gevokizumab) is administered to the subject at about 150 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 175 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 175 mg once every two weeks. In some embodiments, the antibody or antigen binding fragment that specifically binds to IL-1 beta (e.g., canakinumab or gevokizumab) is administered to the subject at about 175 mg once every three weeks. In some embodiments, the antibody or antigen binding fragment that specifically binds to IL-1 beta (e.g., canakinumab or gevokizumab) is administered to the subject at about 175 mg once every four weeks. In some embodiments, the antibody or antigen binding fragment that specifically binds to IL-1 beta (e.g., canakinumab or gevokizumab) is administered to the subject at about 175 mg once every five weeks. In some embodiments, the antibody or antigen binding fragment that specifically binds to IL-1 beta (e.g., canakinumab or gevokizumab) is administered to the subject at about 175 mg once every six weeks. In some embodiments, the antibody or antigen binding fragment that specifically binds to IL-1 beta (e.g., canakinumab or gevokizumab) is administered to the subject at about 175 mg once every seven weeks. In some embodiments, the antibody or antigen binding fragment that specifically binds to IL-1 beta (e.g., canakinumab or gevokizumab) is administered to the subject at about 175 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 200 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 200 mg once every two weeks. In some embodiments, the antibody or antigen binding fragment that specifically binds to IL-1 beta (e.g., canakinumab or gevokizumab) is administered to the subject at about 200 mg once every three weeks. In some embodiments, the antibody or antigen binding fragment that specifically binds to IL-1 beta (e.g., canakinumab or gevokizumab) is administered to the subject at about 200 mg once every four weeks. In some embodiments, the antibody or antigen binding fragment that specifically binds to IL-1 beta (e.g., canakinumab or gevokizumab) is administered to the subject at about 200 mg once every five weeks. In some embodiments, the antibody or antigen binding fragment that specifically binds to IL-1 beta (e.g., canakinumab or gevokizumab) is administered to the subject at about 200 mg once every six weeks. In some embodiments, the antibody or antigen binding fragment that specifically binds to IL-1 beta (e.g., canakinumab or gevokizumab) is administered to the subject at about 200 mg once every seven weeks. In some embodiments, the antibody or antigen binding fragment that specifically binds to IL-1 beta (e.g., canakinumab or gevokizumab) is administered to the subject at about 200 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 225 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 225 mg once every two weeks. In some embodiments, the antibody or antigen binding fragment that specifically binds to IL-1 beta (e.g., canakinumab or gevokizumab) is administered to the subject at about 225 mg once every three weeks. In some embodiments, the antibody or antigen binding fragment that specifically binds to IL-1 beta (e.g., canakinumab or gevokizumab) is administered to the subject at about 225 mg once every four weeks. In some embodiments, the antibody or antigen binding fragment that specifically binds to IL-1 beta (e.g., canakinumab or gevokizumab) is administered to the subject at about 225 mg once every five weeks. In some embodiments, the antibody or antigen binding fragment that specifically binds to IL-1 beta (e.g., canakinumab or gevokizumab) is administered to the subject at about 225 mg once every six weeks. In some embodiments, the antibody or antigen binding fragment that specifically binds to IL-1 beta (e.g., canakinumab or gevokizumab) is administered to the subject at about 225 mg once every seven weeks. In some embodiments, the antibody or antigen binding fragment that specifically binds to IL-1 beta (e.g., canakinumab or gevokizumab) is administered to the subject at about 225 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 250 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 250 mg once every two weeks. In some embodiments, the antibody or antigen binding fragment that specifically binds to IL-1 beta (e.g., canakinumab or gevokizumab) is administered to the subject at about 250 mg once every three weeks. In some embodiments, the antibody or antigen binding fragment that specifically binds to IL-1 beta (e.g., canakinumab or gevokizumab) is administered to the subject at about 250 mg once every four weeks. In some embodiments, the antibody or antigen binding fragment that specifically binds to IL-1 beta (e.g., canakinumab or gevokizumab) is administered to the subject at about 250 mg once every five weeks. In some embodiments, the antibody or antigen binding fragment that specifically binds to IL-1 beta (e.g., canakinumab or gevokizumab) is administered to the subject at about 250 mg once every six weeks. In some embodiments, the antibody or antigen binding fragment that specifically binds to IL-1 beta (e.g., canakinumab or gevokizumab) is administered to the subject at about 250 mg once every seven weeks. In some embodiments, the antibody or antigen binding fragment that specifically binds to IL-1 beta (e.g., canakinumab or gevokizumab) is administered to the subject at about 250 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 275 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 275 mg once every two weeks. In some embodiments, the antibody or antigen binding fragment that specifically binds to IL-1 beta (e.g., canakinumab or gevokizumab) is administered to the subject at about 275 mg once every three weeks. In some embodiments, the antibody or antigen binding fragment that specifically binds to IL-1 beta (e.g., canakinumab or gevokizumab) is administered to the subject at about 275 mg once every four weeks. In some embodiments, the antibody or antigen binding fragment that specifically binds to IL-1 beta (e.g., canakinumab or gevokizumab) is administered to the subject at about 275 mg once every five weeks. In some embodiments, the antibody or antigen binding fragment that specifically binds to IL-1 beta (e.g., canakinumab or gevokizumab) is administered to the subject at about 275 mg once every six weeks. In some embodiments, the antibody or antigen binding fragment that specifically binds to IL-1 beta (e.g., canakinumab or gevokizumab) is administered to the subject at about 275 mg once every seven weeks. In some embodiments, the antibody or antigen binding fragment that specifically binds to IL-1 beta (e.g., canakinumab or gevokizumab) is administered to the subject at about 275 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 300 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 300 mg once every two weeks. In some embodiments, the antibody or antigen binding fragment that specifically binds to IL-1 beta (e.g., canakinumab or gevokizumab) is administered to the subject at about 300 mg once every three weeks. In some embodiments, the antibody or antigen binding fragment that specifically binds to IL-1 beta (e.g., canakinumab or gevokizumab) is administered to the subject at about 300 mg once every four weeks. In some embodiments, the antibody or antigen binding fragment that specifically binds to IL-1 beta (e.g., canakinumab or gevokizumab) is administered to the subject at about 300 mg once every five weeks. In some embodiments, the antibody or antigen binding fragment that specifically binds to IL-1 beta (e.g., canakinumab or gevokizumab) is administered to the subject at about 300 mg once every six weeks. In some embodiments, the antibody or antigen binding fragment that specifically binds to IL-1 beta (e.g., canakinumab or gevokizumab) is administered to the subject at about 300 mg once every seven weeks. In some embodiments, the antibody or antigen binding fragment that specifically binds to IL-1 beta (e.g., canakinumab or gevokizumab) is administered to the subject at about 300 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 325 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 325 mg once every two weeks. In some embodiments, the antibody or antigen binding fragment that specifically binds to IL-1 beta (e.g., canakinumab or gevokizumab) is administered to the subject at about 325 mg once every three weeks. In some embodiments, the antibody or antigen binding fragment that specifically binds to IL-1 beta (e.g., canakinumab or gevokizumab) is administered to the subject at about 325 mg once every four weeks. In some embodiments, the antibody or antigen binding fragment that specifically binds to IL-1 beta (e.g., canakinumab or gevokizumab) is administered to the subject at about 325 mg once every five weeks. In some embodiments, the antibody or antigen binding fragment that specifically binds to IL-1 beta (e.g., canakinumab or gevokizumab) is administered to the subject at about 325 mg once every six weeks. In some embodiments, the antibody or antigen binding fragment that specifically binds to IL-1 beta (e.g., canakinumab or gevokizumab) is administered to the subject at about 325 mg once every seven weeks. In some embodiments, the antibody or antigen binding fragment that specifically binds to IL-1 beta (e.g., canakinumab or gevokizumab) is administered to the subject at about 325 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 350 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 350 mg once every two weeks. In some embodiments, the antibody or antigen binding fragment that specifically binds to IL-1 beta (e.g., canakinumab or gevokizumab) is administered to the subject at about 350 mg once every three weeks. In some embodiments, the antibody or antigen binding fragment that specifically binds to IL-1 beta (e.g., canakinumab or gevokizumab) is administered to the subject at about 350 mg once every four weeks. In some embodiments, the antibody or antigen binding fragment that specifically binds to IL-1 beta (e.g., canakinumab or gevokizumab) is administered to the subject at about 350 mg once every five weeks. In some embodiments, the antibody or antigen binding fragment that specifically binds to IL-1 beta (e.g., canakinumab or gevokizumab) is administered to the subject at about 350 mg once every six weeks. In some embodiments, the antibody or antigen binding fragment that specifically binds to IL-1 beta (e.g., canakinumab or gevokizumab) is administered to the subject at about 350 mg once every seven weeks. In some embodiments, the antibody or antigen binding fragment that specifically binds to IL-1 beta (e.g., canakinumab or gevokizumab) is administered to the subject at about 350 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 375 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 375 mg once every two weeks. In some embodiments, the antibody or antigen binding fragment that specifically binds to IL-1 beta (e.g., canakinumab or gevokizumab) is administered to the subject at about 375 mg once every three weeks. In some embodiments, the antibody or antigen binding fragment that specifically binds to IL-1 beta (e.g., canakinumab or gevokizumab) is administered to the subject at about 375 mg once every four weeks. In some embodiments, the antibody or antigen binding fragment that specifically binds to IL-1 beta (e.g., canakinumab or gevokizumab) is administered to the subject at about 375 mg once every five weeks. In some embodiments, the antibody or antigen binding fragment that specifically binds to IL-1 beta (e.g., canakinumab or gevokizumab) is administered to the subject at about 375 mg once every six weeks. In some embodiments, the antibody or antigen binding fragment that specifically binds to IL-1 beta (e.g., canakinumab or gevokizumab) is administered to the subject at about 375 mg once every seven weeks. In some embodiments, the antibody or antigen binding fragment that specifically binds to IL-1 beta (e.g., canakinumab or gevokizumab) is administered to the subject at about 375 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 400 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 400 mg once every two weeks. In some embodiments, the antibody or antigen binding fragment that specifically binds to IL-1 beta (e.g., canakinumab or gevokizumab) is administered to the subject at about 400 mg once every three weeks. In some embodiments, the antibody or antigen binding fragment that specifically binds to IL-1 beta (e.g., canakinumab or gevokizumab) is administered to the subject at about 400 mg once every four weeks. In some embodiments, the antibody or antigen binding fragment that specifically binds to IL-1 beta (e.g., canakinumab or gevokizumab) is administered to the subject at about 400 mg once every five weeks. In some embodiments, the antibody or antigen binding fragment that specifically binds to IL-1 beta (e.g., canakinumab or gevokizumab) is administered to the subject at about 400 mg once every six weeks. In some embodiments, the antibody or antigen binding fragment that specifically binds to IL-1 beta (e.g., canakinumab or gevokizumab) is administered to the subject at about 400 mg once every seven weeks. In some embodiments, the antibody or antigen binding fragment that specifically binds to IL-1 beta (e.g., canakinumab or gevokizumab) is administered to the subject at about 400 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 425 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 425 mg once every two weeks. In some embodiments, the antibody or antigen binding fragment that specifically binds to IL-1 beta (e.g., canakinumab or gevokizumab) is administered to the subject at about 425 mg once every three weeks. In some embodiments, the antibody or antigen binding fragment that specifically binds to IL-1 beta (e.g., canakinumab or gevokizumab) is administered to the subject at about 425 mg once every four weeks. In some embodiments, the antibody or antigen binding fragment that specifically binds to IL-1 beta (e.g., canakinumab or gevokizumab) is administered to the subject at about 425 mg once every five weeks. In some embodiments, the antibody or antigen binding fragment that specifically binds to IL-1 beta (e.g., canakinumab or gevokizumab) is administered to the subject at about 425 mg once every six weeks. In some embodiments, the antibody or antigen binding fragment that specifically binds to IL-1 beta (e.g., canakinumab or gevokizumab) is administered to the subject at about 425 mg once every seven weeks. In some embodiments, the antibody or antigen binding fragment that specifically binds to IL-1 beta (e.g., canakinumab or gevokizumab) is administered to the subject at about 425 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 450 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 450 mg once every two weeks. In some embodiments, the antibody or antigen binding fragment that specifically binds to IL-1 beta (e.g., canakinumab or gevokizumab) is administered to the subject at about 450 mg once every three weeks. In some embodiments, the antibody or antigen binding fragment that specifically binds to IL-1 beta (e.g., canakinumab or gevokizumab) is administered to the subject at about 450 mg once every four weeks. In some embodiments, the antibody or antigen binding fragment that specifically binds to IL-1 beta (e.g., canakinumab or gevokizumab) is administered to the subject at about 450 mg once every five weeks. In some embodiments, the antibody or antigen binding fragment that specifically binds to IL-1 beta (e.g., canakinumab or gevokizumab) is administered to the subject at about 450 mg once every six weeks. In some embodiments, the antibody or antigen binding fragment that specifically binds to IL-1 beta (e.g., canakinumab or gevokizumab) is administered to the subject at about 450 mg once every seven weeks. In some embodiments, the antibody or antigen binding fragment that specifically binds to IL-1 beta (e.g., canakinumab or gevokizumab) is administered to the subject at about 450 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 475 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 475 mg once every two weeks. In some embodiments, the antibody or antigen binding fragment that specifically binds to IL-1 beta (e.g., canakinumab or gevokizumab) is administered to the subject at about 475 mg once every three weeks. In some embodiments, the antibody or antigen binding fragment that specifically binds to IL-1 beta (e.g., canakinumab or gevokizumab) is administered to the subject at about 475 mg once every four weeks. In some embodiments, the antibody or antigen binding fragment that specifically binds to IL-1 beta (e.g., canakinumab or gevokizumab) is administered to the subject at about 475 mg once every five weeks. In some embodiments, the antibody or antigen binding fragment that specifically binds to IL-1 beta (e.g., canakinumab or gevokizumab) is administered to the subject at about 475 mg once every six weeks. In some embodiments, the antibody or antigen binding fragment that specifically binds to IL-1 beta (e.g., canakinumab or gevokizumab) is administered to the subject at about 475 mg once every seven weeks. In some embodiments, the antibody or antigen binding fragment that specifically binds to IL-1 beta (e.g., canakinumab or gevokizumab) is administered to the subject at about 475 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 500 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 500 mg once every two weeks. In some embodiments, the antibody or antigen binding fragment that specifically binds to IL-1 beta (e.g., canakinumab or gevokizumab) is administered to the subject at about 500 mg once every three weeks. In some embodiments, the antibody or antigen binding fragment that specifically binds to IL-1 beta (e.g., canakinumab or gevokizumab) is administered to the subject at about 500 mg once every four weeks. In some embodiments, the antibody or antigen binding fragment that specifically binds to IL-1 beta (e.g., canakinumab or gevokizumab) is administered to the subject at about 500 mg once every five weeks. In some embodiments, the antibody or antigen binding fragment that specifically binds to IL-1 beta (e.g., canakinumab or gevokizumab) is administered to the subject at about 500 mg once every six weeks. In some embodiments, the antibody or antigen binding fragment that specifically binds to IL-1 beta (e.g., canakinumab or gevokizumab) is administered to the subject at about 500 mg once every seven weeks. In some embodiments, the antibody or antigen binding fragment that specifically binds to IL-1 beta (e.g., canakinumab or gevokizumab) is administered to the subject at about 500 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 525 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 525 mg once every two weeks. In some embodiments, the antibody or antigen binding fragment that specifically binds to IL-1 beta (e.g., canakinumab or gevokizumab) is administered to the subject at about 525 mg once every three weeks. In some embodiments, the antibody or antigen binding fragment that specifically binds to IL-1 beta (e.g., canakinumab or gevokizumab) is administered to the subject at about 525 mg once every four weeks. In some embodiments, the antibody or antigen binding fragment that specifically binds to IL-1 beta (e.g., canakinumab or gevokizumab) is administered to the subject at about 525 mg once every five weeks. In some embodiments, the antibody or antigen binding fragment that specifically binds to IL-1 beta (e.g., canakinumab or gevokizumab) is administered to the subject at about 525 mg once every six weeks. In some embodiments, the antibody or antigen binding fragment that specifically binds to IL-1 beta (e.g., canakinumab or gevokizumab) is administered to the subject at about 525 mg once every seven weeks. In some embodiments, the antibody or antigen binding fragment that specifically binds to IL-1 beta (e.g., canakinumab or gevokizumab) is administered to the subject at about 525 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 550 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 550 mg once every two weeks. In some embodiments, the antibody or antigen binding fragment that specifically binds to IL-1 beta (e.g., canakinumab or gevokizumab) is administered to the subject at about 550 mg once every three weeks. In some embodiments, the antibody or antigen binding fragment that specifically binds to IL-1 beta (e.g., canakinumab or gevokizumab) is administered to the subject at about 550 mg once every four weeks. In some embodiments, the antibody or antigen binding fragment that specifically binds to IL-1 beta (e.g., canakinumab or gevokizumab) is administered to the subject at about 550 mg once every five weeks. In some embodiments, the antibody or antigen binding fragment that specifically binds to IL-1 beta (e.g., canakinumab or gevokizumab) is administered to the subject at about 550 mg once every six weeks. In some embodiments, the antibody or antigen binding fragment that specifically binds to IL-1 beta (e.g., canakinumab or gevokizumab) is administered to the subject at about 550 mg once every seven weeks. In some embodiments, the antibody or antigen binding fragment that specifically binds to IL-1 beta (e.g., canakinumab or gevokizumab) is administered to the subject at about 550 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 575 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 575 mg once every two weeks. In some embodiments, the antibody or antigen binding fragment that specifically binds to IL-1 beta (e.g., canakinumab or gevokizumab) is administered to the subject at about 575 mg once every three weeks. In some embodiments, the antibody or antigen binding fragment that specifically binds to IL-1 beta (e.g., canakinumab or gevokizumab) is administered to the subject at about 575 mg once every four weeks. In some embodiments, the antibody or antigen binding fragment that specifically binds to IL-1 beta (e.g., canakinumab or gevokizumab) is administered to the subject at about 575 mg once every five weeks. In some embodiments, the antibody or antigen binding fragment that specifically binds to IL-1 beta (e.g., canakinumab or gevokizumab) is administered to the subject at about 575 mg once every six weeks. In some embodiments, the antibody or antigen binding fragment that specifically binds to IL-1 beta (e.g., canakinumab or gevokizumab) is administered to the subject at about 575 mg once every seven weeks. In some embodiments, the antibody or antigen binding fragment that specifically binds to IL-1 beta (e.g., canakinumab or gevokizumab) is administered to the subject at about 575 mg once every eight weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at about 600 mg once every two weeks, every three weeks, every four weeks, every five weeks, every six weeks, every seven weeks, every eight weeks, every nine weeks, every ten weeks, every eleven weeks, or every twelve weeks in order to reduce the severity and/or duration of one or more manifestation(s) or complication(s) of sickle cell disease described herein. In some embodiments, the antibody or antigen binding fragment that specifically binds to IL-1 beta (e.g., canakinumab or gevokizumab) is administered to the subject at about 600 mg once every two weeks. In some embodiments, the antibody or antigen binding fragment that specifically binds to IL-1 beta (e.g., canakinumab or gevokizumab) is administered to the subject at about 600 mg once every three weeks. In some embodiments, the antibody or antigen binding fragment that specifically binds to IL-1 beta (e.g., canakinumab or gevokizumab) is administered to the subject at about 600 mg once every four weeks. In some embodiments, the antibody or antigen binding fragment that specifically binds to IL-1 beta (e.g., canakinumab or gevokizumab) is administered to the subject at about 600 mg once every five weeks. In some embodiments, the antibody or antigen binding fragment that specifically binds to IL-1 beta (e.g., canakinumab or gevokizumab) is administered to the subject at about 600 mg once every six weeks. In some embodiments, the antibody or antigen binding fragment that specifically binds to IL-1 beta (e.g., canakinumab or gevokizumab) is administered to the subject at about 600 mg once every seven weeks. In some embodiments, the antibody or antigen binding fragment that specifically binds to IL-1 beta (e.g., canakinumab or gevokizumab) is administered to the subject at about 600 mg once every eight weeks.

In one embodiment, about 30 mg to about 240 mg gevokizumab is administered to the patient every four weeks, every six weeks, every eight weeks, or every twelve weeks. In one embodiment gevokizumab is administered subcutaneously. In one embodiment gevokizumab is preferably administered intravenously. In one embodiment, gevokizumab is administered to the patient at about 30 mg every two weeks, every three weeks, every four weeks, every eight weeks, or every twelve weeks. In one embodiment, gevokizumab is administered to the patient at about 60 mg every two weeks, every three weeks, every four weeks, every eight weeks, or every twelve weeks. In one embodiment, gevokizumab is administered to the patient at about 90 mg every two weeks, every three weeks, every four weeks, every eight weeks, or every twelve weeks. In one embodiment, gevokizumab is administered to the patient at about 120 mg every two weeks, every three weeks, every four weeks, every eight weeks, or every twelve weeks. In one embodiment, gevokizumab is administered to the patient at about 180 mg every two weeks, every three weeks, every four weeks, every eight weeks, or every twelve weeks. In one embodiment, gevokizumab is administered to the patient at about 240 mg every two weeks, every three weeks, every four weeks, every eight weeks, or every twelve weeks.

In some embodiments, provided herein is a method of treating, preventing, reducing, or eliminating a manifestation or complication associated with sickle cell disease in a subject in need thereof, the method comprising administering to the subject a therapeutically effective amount of an antibody or antigen binding fragment that specifically binds to IL-1 beta (e.g., canakinumab or gevokizumab), wherein the antibody or antigen binding fragment that specifically binds to IL-1 beta is administered to a subject with sickle cell disease at an initial dose and, subsequently, a maintenance dose. In some embodiments the initial dose is about 400 mg to about 600 mg (e.g., about 400 mg, about 425 mg, about 450 mg, about 475 mg, about 500 mg, about 525 mg, about 550 mg, about 575 mg, or about 600 mg) once every two weeks, once every three weeks, or once every four weeks. In some embodiments the initial dose is about 300 mg to about 600 mg (e.g., about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg) once every two weeks, once every three weeks, or once every four weeks. In some embodiments the initial dose is about 300 mg to about 500 mg (e.g., about 300 mg, about 325 mg, about 350 mg, about 375 mg, about 400 mg, about 425 mg, about 450 mg, about 475 mg, or about 500 mg) once every two weeks, once every three weeks, or once every four weeks. In some embodiments, the maintenance dose is lower than the initial dose, e.g., about 100 mg, about 150 mg to about 300 mg, about 200 mg to about 350 mg, about 250 mg to about 400 mg, or about 300 mg to about 450 mg. As a set of non-limiting examples, the maintenance dose may be about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, or about 550 mg. Such administrations of the anti-interleukin 1 beta antibody (e.g., canakinumab or gevokizumab) to the subject with sickle cell disease may occur, e.g., every two weeks, every three weeks, every four weeks, every five weeks, every six weeks, every seven weeks, every eight weeks, every nine weeks, every ten weeks, every eleven weeks, or every twelve weeks.

In some embodiments, the initial dose may be about 150 mg to about 200 mg and the maintenance dose may be about 150 mg. In some embodiments, the initial dose may be about 200 mg to about 250 mg and the maintenance dose may be about 150 mg. In some embodiments, the initial dose may be about 200 mg to about 250 mg and the maintenance dose may be about 200 mg. In some embodiments, the initial dose may be about 250 mg to about 300 mg and the maintenance dose may be about 200 mg. In some embodiments, the initial dose may be about 250 mg to about 300 mg and the maintenance dose may be about 150 mg. In some embodiments, the initial dose may be about 250 mg to about 300 mg and the maintenance dose may be about 250 mg. In some embodiments, the initial dose may be about 250 mg to about 350 mg and the maintenance dose may be about 200 mg. In some embodiments, the initial dose may be about 250 mg to about 350 mg and the maintenance dose may be about 150 mg. In some embodiments, the initial dose may be about 250 mg to about 350 mg and the maintenance dose may be about 250 mg. In some embodiments, the initial dose may be about 250 mg to about 350 mg and the maintenance dose may be about 300 mg. Such initial doses may be given once, twice, three times, four times, five times, or six times and may be given every two weeks, every three weeks, every four weeks, or every five weeks. Such initial doses are administered in a period termed the “loading phase” or the “initial phase”. In some embodiments, the loading phase is at least 2 months, at least 3 months, at least four months, at least five months, or at least six months. Such maintenance doses may be given in a period known as the “maintenance phase” subsequently to the planned initial doses (i.e., after the initial or leading phase) and may be given, for example, every three weeks, every four weeks, every five weeks, every six weeks, every seven weeks, every eight weeks, every nine weeks, every ten weeks, every eleven weeks, or every twelve weeks.

In some embodiments, the IL-1 beta binding antibody or antigen binding fragment thereof may be administered to the subject in combination with crizanlizumab on the same day, optionally not more than 2 hours apart or not more than one hour apart. In some embodiments, the antibody or antigen binding fragment thereof that specifically binds to IL-1 beta is administered to the subject in a loading phase followed by a maintenance phase as described herein, wherein the subject receives a higher amount of the antibody during the loading phase than during the maintenance phase over the same given period of time. In some embodiments, the loading phase is at least 2 months. In some embodiments, the loading phase is at least 3 months. In some embodiments, the loading phase is at least 4 months. In some embodiments, the loading phase is at least 5 months. In some embodiments, the loading phase is at least 6 months. In some embodiments, the administration interval within the loading phase is the same as that within the maintenance phase. In some embodiments, the dose within the loading phase (loading dose) is at least twice the amount of the dose within the maintenance phase (maintenance dose). In some embodiments, the loading dose is the same as the maintenance dose. In some embodiments, the administration interval within the maintenance phase is twice or three times as long as the interval within the loading phase.

In some embodiments, canakinumab is administered at 300 mg during the loading phase and 150 mg during the maintenance phase. In some embodiments, canakinumab is administered at 400 mg during the loading phase and 200 mg during the maintenance phase. In some embodiments, canakinumab is administered at 500 mg during the loading phase and 250 mg during the maintenance phase. In some embodiments, canakinumab is administered at 600 mg during the loading phase and 300 mg during the maintenance phase. In some embodiments, gevokizumab is administered at 180 mg during the loading phase and 90 mg during the maintenance phase. In some embodiments, gevokizumab is administered at 120 mg during the loading phase and 60 mg during the maintenance phase. In some embodiments, gevokizumab is administered at 500 mg during the loading phase and 250 mg during the maintenance phase. In some embodiments, gevokizumab is administered at 60 mg during the loading phase and 30 mg during the maintenance phase.

In some embodiments, the administration interval (time between doses) is longer within the maintenance phase than within the loading phase. In one embodiment, the administration interval of the maintenance phase is twice as long as the administration interval of the loading phase. In one embodiment, the administration interval of the maintenance phase is three times as long as the administration interval of the loading phase. In one embodiment, the administration interval of the maintenance phase is four times as long as the administration interval of the loading phase. In some embodiments, the loading dose may be administered every two weeks and the maintenance dose may be administered every four or six weeks. In some embodiments, the loading dose may be administered every three weeks and the maintenance dose may be administered every six or nine weeks. In some embodiments, the loading dose may be administered every four weeks and the maintenance dose may be administered every two or three months. In such embodiments, the dose of canakinumab may be selected from, e.g., 150 mg, 200 mg, 250 mg, 300 mg, 350 mg, or 400 mg.

An initial dose or loading dose may be first administered to a subject to, e.g., resolve a crisis, exigent, or emergent symptom. Such an initial dose or loading dose may be, e.g., of a greater amount and/or administered more frequently than a maintenance dose (administered to the patient on an ongoing basis in order to continue relief from or reduction in symptoms). Such an initial dose may be administered until, for example, there is reduction in severity and/or frequency of one or more of the manifestations or complications described herein (e.g., for sickle cell disease).

Over time, dosages may increase or decrease as considered necessary for relief or abatement of symptoms by a medical professional directing the care of the subject. Intervals between doses may likewise increase or decrease as considered necessary for relief or abatement of one or more of the manifestations or complications described herein (e.g., for sickle cell disease) by a medical professional directing the care of the subject. Specifically, during the maintenance phase, an ad hoc dose may be administered at a higher amount than the normal maintenance dose or at a shorter interval than the normal maintenance interval for that patient in response to acute flares of sickle cell disease (e.g., in response to one or more manifestations or complications of sickle disease such as those described herein) or may be adminstered prophylactically before anticipated stressful events.

All the aforementioned embodiments for the methods of protection and treatment according to the present invention are equally applicable to
the use of any one of the antibodies or antigen binding fragments as described herein for the manufacture of a medicament for use according to the present invention,
the use of any one of the antibodies or antigen binding fragments described herein according to the present invention,
any one of the antibodies or antigen binding fragments described herein for use according to the present invention,
the pharmaceutical compositions comprising any one of the antibodies or antigen binding fragments described herein for the use according to the present invention,
the use of the pharmaceutical compositions comprising any one of the antibodies or antigen binding fragments described herein according to the present invention, and
the use of the pharmaceutical compositions comprising any one of the antibodies or antigen binding fragments described herein for the manufacture of a medicament for use according to the present invention.

Combination Therapies

The various treatments described above can be combined with other treatment partners or therapeutic agents such as the current standard of care for a disease associated with sickle cell disease. For example, the anti-interleukin 1 beta antibody (e.g., canakinumab or gevokizumab) can be combined with one or more of an antibody or antigen binding fragment thereof that specifically binds to p selectin, L-glutamine oral powder, an agent that increases fetal hemoglobin, and combinations thereof. In some embodiments, the agent that increases fetal hemoglobin is hydroxyurea, an antibody or antigen binding fragment thereof that specifically binds to p selectin, L-glutamine oral powder, voxelotor, or stem cells (e.g., blood-producing hematopietic stem cells (HSCs)) comprising a lentiviral vector which inserts a functioning version of the HBB or the HBF gene gene (e.g., Zynteglo or LentiGlobin BB305). In some embodiments, the subject has already been treated with one or more gene replacement or gene editing therapies (e.g., HGB-206, CTX-001, ST-100, or RVT-1801) but may still have one or more manifestations or complications of sickle cell disease (e.g., one or more of the manifestations or complications described herein). In such cases, the subject may additionally benefit from one or more of the treatment regimens described herein. As another set of non-limiting examples, the anti-interleukin 1 beta antibody (e.g., canakinumab or gevokizumab) can be combined with or or more of: an IL-18 inhibitor and/or other inflammasome pathway inhibitor (e.g., inhibitors of NLRP3, gasdermin D, or other inflammasome pathway members), an agent preventing red cell alterations (e.g., Hb polymerization, dehydration, microparticle generation, or mobilization of Weibel-Palade bodies), an ATP or ATP receptor inhibitor, a Bach1 inhibitor, a CD40 pathway inhibitor, a P-selectin pathway inhibitor, an inhibitors of an adhesion molecule (e.g., ICAM or VCAM), a compound preventing platelet activation, a platelet stabilizing compound (e.g., multimerized IgG1Fc or IVIG), a TLR antagonist (e.g., an antagonist of TLR4, TLR7, TLR8, and/or TRL9), a ROS inhibitor, an agent for regulation of oxygen regulated genes (e.g., HIF2a), an agent that prevents or disrupts NET formation, a complement inhibitor, a heme oxygenase-1/HO-lpathway modulator, a CXCR4 pathway modulator, a phosphodiesterase inhibitor, and agent that acts in an anti-angiogenic manner, EPO, a leukotriene inhibitors (e.g., Leukotriene A-4 hydrolase), or combinations thereof.

In some embodiments, the anti-interleukin 1 beta antibody (e.g., canakinumab or gevokizumab) at any of the amounts described herein may be combined with hydroxyurea (e.g., wherein the hydroxyurea is administered to the patient at about 10 mg/kg/day to about 35 mg/kg/day, for example at about 10 mg/kg/day, about 15 mg/kg/day, about 20 mg/kg/day, about 25 mg/kg/day, about 30 mg/kg/day, or about 35 mg/kg/day).

In some embodiments, the anti-interleukin 1 beta antibody (e.g., canakinumab or gevokizumab) or antigen binding fragment thereof may be combined with hydroxyurea. In some embodiments, the anti-interleukin 1 beta antibody (e.g., canakinumab or gevokizumab) or antigen binding fragment thereof may be administered at any of the amounts described herein in combination with hydroxyurea. As a set of non-limiting examples, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea. As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea at about 10 mg/kg/day to about 35 mg/kg/day, for example at about 10 mg/kg/day, about 15 mg/kg/day, about 20 mg/kg/day, about 25 mg/kg/day, about 30 mg/kg/day, or about 35 mg/kg/day). As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea at about 10 mg/kg/day to about 35 mg/kg/day, for example at about 10 mg/kg/day, about 15 mg/kg/day, about 20 mg/kg/day, about 25 mg/kg/day, about 30 mg/kg/day, or about 35 mg/kg/day). In some embodiments, the anti-interleukin 1 beta antibody (e.g., canakinumab or gevokizumab) may be administered to the subject every two weeks while the hydroxyurea is administered daily. In some embodiments, the anti-interleukin 1 beta antibody (e.g., canakinumab or gevokizumab) may be administered to the subject every three weeks while the hydroxyurea is administered daily. In some embodiments, the anti-interleukin 1 beta antibody (e.g., canakinumab or gevokizumab) may be administered to the subject every four weeks while the hydroxyurea is administered daily. In some embodiments, the anti-interleukin 1 beta antibody (e.g., canakinumab or gevokizumab) may be administered to the subject every five weeks while the hydroxyurea is administered daily.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea at about 10 mg/kg/day. As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea at about 10 mg/kg/day. In some embodiments, the anti-interleukin 1 beta antibody (e.g., canakinumab or gevokizumab) may be administered to the subject every two weeks while the hydroxyurea is administered daily. In some embodiments, the anti-interleukin 1 beta antibody (e.g., canakinumab or gevokizumab) may be administered to the subject every three weeks while the hydroxyurea is administered daily. In some embodiments, the anti-interleukin 1 beta antibody (e.g., canakinumab or gevokizumab) may be administered to the subject every four weeks while the hydroxyurea is administered daily. In some embodiments, the anti-interleukin 1 beta antibody (e.g., canakinumab or gevokizumab) may be administered to the subject every five weeks while the hydroxyurea is administered daily.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea at about 15 mg/kg/day. As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea at about 15 mg/kg/day. In some embodiments, the anti-interleukin 1 beta antibody (e.g., canakinumab or gevokizumab) may be administered to the subject every two weeks while the hydroxyurea is administered daily. In some embodiments, the anti-interleukin 1 beta antibody (e.g., canakinumab or gevokizumab) may be administered to the subject every three weeks while the hydroxyurea is administered daily. In some embodiments, the anti-interleukin 1 beta antibody (e.g., canakinumab or gevokizumab) may be administered to the subject every four weeks while the hydroxyurea is administered daily. In some embodiments, the anti-interleukin 1 beta antibody (e.g., canakinumab or gevokizumab) may be administered to the subject every five weeks while the hydroxyurea is administered daily.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea at about 20 mg/kg/day. As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea at about 20 mg/kg/day. In some embodiments, the anti-interleukin 1 beta antibody (e.g., canakinumab or gevokizumab) may be administered to the subject every two weeks while the hydroxyurea is administered daily. In some embodiments, the anti-interleukin 1 beta antibody (e.g., canakinumab or gevokizumab) may be administered to the subject every three weeks while the hydroxyurea is administered daily. In some embodiments, the anti-interleukin 1 beta antibody (e.g., canakinumab or gevokizumab) may be administered to the subject every four weeks while the hydroxyurea is administered daily. In some embodiments, the anti-interleukin 1 beta antibody (e.g., canakinumab or gevokizumab) may be administered to the subject every five weeks while the hydroxyurea is administered daily.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea at about 25 mg/kg/day. As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea at about 25 mg/kg/day. In some embodiments, the anti-interleukin 1 beta antibody (e.g., canakinumab or gevokizumab) may be administered to the subject every two weeks while the hydroxyurea is administered daily. In some embodiments, the anti-interleukin 1 beta antibody (e.g., canakinumab or gevokizumab) may be administered to the subject every three weeks while the hydroxyurea is administered daily. In some embodiments, the anti-interleukin 1 beta antibody (e.g., canakinumab or gevokizumab) may be administered to the subject every four weeks while the hydroxyurea is administered daily. In some embodiments, the anti-interleukin 1 beta antibody (e.g., canakinumab or gevokizumab) may be administered to the subject every five weeks while the hydroxyurea is administered daily.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea at about 30 mg/kg/day. As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea at about 30 mg/kg/day. In some embodiments, the anti-interleukin 1 beta antibody (e.g., canakinumab or gevokizumab) may be administered to the subject every two weeks while the hydroxyurea is administered daily. In some embodiments, the anti-interleukin 1 beta antibody (e.g., canakinumab or gevokizumab) may be administered to the subject every three weeks while the hydroxyurea is administered daily. In some embodiments, the anti-interleukin 1 beta antibody (e.g., canakinumab or gevokizumab) may be administered to the subject every four weeks while the hydroxyurea is administered daily. In some embodiments, the anti-interleukin 1 beta antibody (e.g., canakinumab or gevokizumab) may be administered to the subject every five weeks while the hydroxyurea is administered daily.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, or about 300 mg to about 450 mg in combination with hydroxyurea at about 35 mg/kg/day. As a set of non-limiting examples, the anti-interleukin 1 beta antibody (e.g., canakinumab or gevokizumab) or antigen binding fragment thereof may be administered to a subject with sickle cell disease at about 150 mg, about 175 mg, about 200 mg, about 225 mg, about 250 mg, about 275 mg, about 300 mg, about 325 mg, about 350 mg, about 375 mg, or about 400 mg in combination with hydroxyurea at about 35 mg/kg/day. In some embodiments, the anti-interleukin 1 beta antibody (e.g., canakinumab or gevokizumab) may be administered to the subject every two weeks while the hydroxyurea is administered daily. In some embodiments, the anti-interleukin 1 beta antibody (e.g., canakinumab or gevokizumab) may be administered to the subject every three weeks while the hydroxyurea is administered daily. In some embodiments, the anti-interleukin 1 beta antibody (e.g., canakinumab or gevokizumab) may be administered to the subject every four weeks while the hydroxyurea is administered daily. In some embodiments, the anti-interleukin 1 beta antibody (e.g., canakinumab or gevokizumab) may be administered to the subject every five weeks while the hydroxyurea is administered daily.

In some embodiments, the anti-interleukin 1 beta antibody (e.g., canakinumab or gevokizumab) or antigen binding fragment thereof may be combined with an antibody that specifically binds to p selectin (e.g., crizanlizumab) or antigen binding fragment thereof. In some embodiments, the the anti-interleukin 1 beta antibody (e.g., canakinumab or gevokizumab) or antigen binding fragment thereof may be administered at any of the amounts described herein in combination with an antibody that specifically binds to p selectin (e.g., crizanlizumab) or antigen binding fragment thereof. As a set of non-limiting examples, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, or about 500 mg to about 600 mg in combination with the antibody that specifically binds to p selectin (e.g., crizanlizumab) or antigen binding fragment thereof. As a set of non-limiting examples, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 2.5 to about 5 mg/kg. As a set of non-limiting examples, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 2.5 mg/kg, about 3 mg/kg, about 3.5 mg/kg, about 4 mg/kg, about 4.5 mg/kg, or about 5 mg/kg. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every two weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every three weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every four weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every five weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 2.5 to about 5 mg/kg. As a set of non-limiting examples, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 2.5 mg/kg, about 3 mg/kg, about 3.5 mg/kg, about 4 mg/kg, about 4.5 mg/kg, or about 5 mg/kg. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every two weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every three weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every four weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every five weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 4 to about 7.5 mg/kg. As a set of non-limiting examples, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4 mg/kg, about 4.5 mg/kg, about 5 mg/kg, about 5.5 mg/kg, about 6 mg/kg, about 6.5 mg/kg, about 7 mg/kg, or about 5 mg/kg. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every two weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every three weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every four weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every five weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 2.5 to about 5 mg/kg, wherein the combination is administered to the subject about every two weeks. In some embodiments, the anti-interleukin 1 beta antibody (e.g., canakinumab or gevokizumab) may be administered to a subject with sickle cell disease at about 150 mg to about 300 mg in combination with the antibody that specifically binds to p selectin (e.g., crizanlizumab) or antigen binding fragment thereof at about 2.5 to about 5 mg/kg, wherein the combination is administered to the subject about every two weeks. In some embodiments, the anti-interleukin 1 beta antibody (e.g., canakinumab or gevokizumab) may be administered to a subject with sickle cell disease at about about 200 mg to about 350 mg in combination with the antibody that specifically binds to p selectin (e.g., crizanlizumab) or antigen binding fragment thereof at about 2.5 to about 5 mg/kg, wherein the combination is administered to the subject about every two weeks. In some embodiments, the anti-interleukin 1 beta antibody (e.g., canakinumab or gevokizumab) may be administered to a subject with sickle cell disease at about about 250 mg to about 400 mg in combination with the antibody that specifically binds to p selectin (e.g., crizanlizumab) or antigen binding fragment thereof at about 2.5 to about 5 mg/kg, wherein the combination is administered to the subject about every two weeks. In some embodiments, the anti-interleukin 1 beta antibody (e.g., canakinumab or gevokizumab) may be administered to a subject with sickle cell disease at about about 300 mg to about 450 mg in combination with the antibody that specifically binds to p selectin (e.g., crizanlizumab) or antigen binding fragment thereof at about 2.5 to about 5 mg/kg, wherein the combination is administered to the subject about every two weeks. In some embodiments, the anti-interleukin 1 beta antibody (e.g., canakinumab or gevokizumab) may be administered to a subject with sickle cell disease at about about 350 mg to about 500 mg in combination with the antibody that specifically binds to p selectin (e.g., crizanlizumab) or antigen binding fragment thereof at about 2.5 to about 5 mg/kg, wherein the combination is administered to the subject about every two weeks. In some embodiments, the anti-interleukin 1 beta antibody (e.g., canakinumab or gevokizumab) may be administered to a subject with sickle cell disease at about about 400 mg to about 550 mg in combination with the antibody that specifically binds to p selectin (e.g., crizanlizumab) or antigen binding fragment thereof at about 2.5 to about 5 mg/kg, wherein the combination is administered to the subject about every two weeks. In some embodiments, the anti-interleukin 1 beta antibody (e.g., canakinumab or gevokizumab) may be administered to a subject with sickle cell disease at about about 500 mg to about 600 mg in combination with the antibody that specifically binds to p selectin (e.g., crizanlizumab) or antigen binding fragment thereof at about 2.5 to about 5 mg/kg, wherein the combination is administered to the subject about every two weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 2.5 to about 5 mg/kg, wherein the combination is administered to the subject about every three weeks. In some embodiments, the anti-interleukin 1 beta antibody (e.g., canakinumab or gevokizumab) may be administered to a subject with sickle cell disease at about 150 mg to about 300 mg in combination with the antibody that specifically binds to p selectin (e.g., crizanlizumab) or antigen binding fragment thereof at about 2.5 to about 5 mg/kg, wherein the combination is administered to the subject about every three weeks. In some embodiments, the anti-interleukin 1 beta antibody (e.g., canakinumab or gevokizumab) may be administered to a subject with sickle cell disease at about about 200 mg to about 350 mg in combination with the antibody that specifically binds to p selectin (e.g., crizanlizumab) or antigen binding fragment thereof at about 2.5 to about 5 mg/kg, wherein the combination is administered to the subject about every three weeks. In some embodiments, the anti-interleukin 1 beta antibody (e.g., canakinumab or gevokizumab) may be administered to a subject with sickle cell disease at about about 250 mg to about 400 mg in combination with the antibody that specifically binds to p selectin (e.g., crizanlizumab) or antigen binding fragment thereof at about 2.5 to about 5 mg/kg, wherein the combination is administered to the subject about every three weeks. In some embodiments, the anti-interleukin 1 beta antibody (e.g., canakinumab or gevokizumab) may be administered to a subject with sickle cell disease at about about 300 mg to about 450 mg in combination with the antibody that specifically binds to p selectin (e.g., crizanlizumab) or antigen binding fragment thereof at about 2.5 to about 5 mg/kg, wherein the combination is administered to the subject about every three weeks. In some embodiments, the anti-interleukin 1 beta antibody (e.g., canakinumab or gevokizumab) may be administered to a subject with sickle cell disease at about about 350 mg to about 500 mg in combination with the antibody that specifically binds to p selectin (e.g., crizanlizumab) or antigen binding fragment thereof at about 2.5 to about 5 mg/kg, wherein the combination is administered to the subject about every three weeks. In some embodiments, the anti-interleukin 1 beta antibody (e.g., canakinumab or gevokizumab) may be administered to a subject with sickle cell disease at about about 400 mg to about 550 mg in combination with the antibody that specifically binds to p selectin (e.g., crizanlizumab) or antigen binding fragment thereof at about 2.5 to about 5 mg/kg, wherein the combination is administered to the subject about every three weeks. In some embodiments, the anti-interleukin 1 beta antibody (e.g., canakinumab or gevokizumab) may be administered to a subject with sickle cell disease at about about 500 mg to about 600 mg in combination with the antibody that specifically binds to p selectin (e.g., crizanlizumab) or antigen binding fragment thereof at about 2.5 to about 5 mg/kg, wherein the combination is administered to the subject about every three weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 2.5 to about 5 mg/kg, wherein the combination is administered to the subject about every four weeks. In some embodiments, the anti-interleukin 1 beta antibody (e.g., canakinumab or gevokizumab) may be administered to a subject with sickle cell disease at about 150 mg to about 300 mg in combination with the antibody that specifically binds to p selectin (e.g., crizanlizumab) or antigen binding fragment thereof at about 2.5 to about 5 mg/kg, wherein the combination is administered to the subject about every four weeks. In some embodiments, the anti-interleukin 1 beta antibody (e.g., canakinumab or gevokizumab) may be administered to a subject with sickle cell disease at about about 200 mg to about 350 mg in combination with the antibody that specifically binds to p selectin (e.g., crizanlizumab) or antigen binding fragment thereof at about 2.5 to about 5 mg/kg, wherein the combination is administered to the subject about every four weeks. In some embodiments, the anti-interleukin 1 beta antibody (e.g., canakinumab or gevokizumab) may be administered to a subject with sickle cell disease at about about 250 mg to about 400 mg in combination with the antibody that specifically binds to p selectin (e.g., crizanlizumab) or antigen binding fragment thereof at about 2.5 to about 5 mg/kg, wherein the combination is administered to the subject about every four weeks. In some embodiments, the anti-interleukin 1 beta antibody (e.g., canakinumab or gevokizumab) may be administered to a subject with sickle cell disease at about about 300 mg to about 450 mg in combination with the antibody that specifically binds to p selectin (e.g., crizanlizumab) or antigen binding fragment thereof at about 2.5 to about 5 mg/kg, wherein the combination is administered to the subject about every four weeks. In some embodiments, the anti-interleukin 1 beta antibody (e.g., canakinumab or gevokizumab) may be administered to a subject with sickle cell disease at about about 350 mg to about 500 mg in combination with the antibody that specifically binds to p selectin (e.g., crizanlizumab) or antigen binding fragment thereof at about 2.5 to about 5 mg/kg, wherein the combination is administered to the subject about every four weeks. In some embodiments, the anti-interleukin 1 beta antibody (e.g., canakinumab or gevokizumab) may be administered to a subject with sickle cell disease at about about 400 mg to about 550 mg in combination with the antibody that specifically binds to p selectin (e.g., crizanlizumab) or antigen binding fragment thereof at about 2.5 to about 5 mg/kg, wherein the combination is administered to the subject about every four weeks. In some embodiments, the anti-interleukin 1 beta antibody (e.g., canakinumab or gevokizumab) may be administered to a subject with sickle cell disease at about about 500 mg to about 600 mg in combination with the antibody that specifically binds to p selectin (e.g., crizanlizumab) or antigen binding fragment thereof at about 2.5 to about 5 mg/kg, wherein the combination is administered to the subject about every four weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 4 to about 7.5 mg/kg, wherein the combination is administered to the subject about every two weeks. In some embodiments, the anti-interleukin 1 beta antibody (e.g., canakinumab or gevokizumab) may be administered to a subject with sickle cell disease at about 150 mg to about 300 mg in combination with the antibody that specifically binds to p selectin (e.g., crizanlizumab) or antigen binding fragment thereof at about 4 to about 7.5 mg/kg, wherein the combination is administered to the subject about every two weeks. In some embodiments, the anti-interleukin 1 beta antibody (e.g., canakinumab or gevokizumab) may be administered to a subject with sickle cell disease at about about 200 mg to about 350 mg in combination with the antibody that specifically binds to p selectin (e.g., crizanlizumab) or antigen binding fragment thereof at about 4 to about 7.5 mg/kg, wherein the combination is administered to the subject about every two weeks. In some embodiments, the anti-interleukin 1 beta antibody (e.g., canakinumab or gevokizumab) may be administered to a subject with sickle cell disease at about about 250 mg to about 400 mg in combination with the antibody that specifically binds to p selectin (e.g., crizanlizumab) or antigen binding fragment thereof at about 4 to about 7.5 mg/kg, wherein the combination is administered to the subject about every two weeks. In some embodiments, the anti-interleukin 1 beta antibody (e.g., canakinumab or gevokizumab) may be administered to a subject with sickle cell disease at about about 300 mg to about 450 mg in combination with the antibody that specifically binds to p selectin (e.g., crizanlizumab) or antigen binding fragment thereof at about 4 to about 7.5 mg/kg, wherein the combination is administered to the subject about every two weeks. In some embodiments, the anti-interleukin 1 beta antibody (e.g., canakinumab or gevokizumab) may be administered to a subject with sickle cell disease at about about 350 mg to about 500 mg in combination with the antibody that specifically binds to p selectin (e.g., crizanlizumab) or antigen binding fragment thereof at about 4 to about 7.5 mg/kg, wherein the combination is administered to the subject about every two weeks. In some embodiments, the anti-interleukin 1 beta antibody (e.g., canakinumab or gevokizumab) may be administered to a subject with sickle cell disease at about about 400 mg to about 550 mg in combination with the antibody that specifically binds to p selectin (e.g., crizanlizumab) or antigen binding fragment thereof at about 4 to about 7.5 mg/kg, wherein the combination is administered to the subject about every two weeks. In some embodiments, the anti-interleukin 1 beta antibody (e.g., canakinumab or gevokizumab) may be administered to a subject with sickle cell disease at about about 500 mg to about 600 mg in combination with the antibody that specifically binds to p selectin (e.g., crizanlizumab) or antigen binding fragment thereof at about 4 to about 7.5 mg/kg, wherein the combination is administered to the subject about every two weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 4 to about 7.5 mg/kg, wherein the combination is administered to the subject about every three weeks. In some embodiments, the anti-interleukin 1 beta antibody (e.g., canakinumab or gevokizumab) may be administered to a subject with sickle cell disease at about 150 mg to about 300 mg in combination with the antibody that specifically binds to p selectin (e.g., crizanlizumab) or antigen binding fragment thereof at about 4 to about 7.5 mg/kg, wherein the combination is administered to the subject about every three weeks. In some embodiments, the anti-interleukin 1 beta antibody (e.g., canakinumab or gevokizumab) may be administered to a subject with sickle cell disease at about about 200 mg to about 350 mg in combination with the antibody that specifically binds to p selectin (e.g., crizanlizumab) or antigen binding fragment thereof at about 4 to about 7.5 mg/kg, wherein the combination is administered to the subject about every three weeks. In some embodiments, the anti-interleukin 1 beta antibody (e.g., canakinumab or gevokizumab) may be administered to a subject with sickle cell disease at about about 250 mg to about 400 mg in combination with the antibody that specifically binds to p selectin (e.g., crizanlizumab) or antigen binding fragment thereof at about 4 to about 7.5 mg/kg, wherein the combination is administered to the subject about every three weeks. In some embodiments, the anti-interleukin 1 beta antibody (e.g., canakinumab or gevokizumab) may be administered to a subject with sickle cell disease at about about 300 mg to about 450 mg in combination with the antibody that specifically binds to p selectin (e.g., crizanlizumab) or antigen binding fragment thereof at about 4 to about 7.5 mg/kg, wherein the combination is administered to the subject about every three weeks. In some embodiments, the anti-interleukin 1 beta antibody (e.g., canakinumab or gevokizumab) may be administered to a subject with sickle cell disease at about about 350 mg to about 500 mg in combination with the antibody that specifically binds to p selectin (e.g., crizanlizumab) or antigen binding fragment thereof at about 4 to about 7.5 mg/kg, wherein the combination is administered to the subject about every three weeks. In some embodiments, the anti-interleukin 1 beta antibody (e.g., canakinumab or gevokizumab) may be administered to a subject with sickle cell disease at about about 400 mg to about 550 mg in combination with the antibody that specifically binds to p selectin (e.g., crizanlizumab) or antigen binding fragment thereof at about 4 to about 7.5 mg/kg, wherein the combination is administered to the subject about every three weeks. In some embodiments, the anti-interleukin 1 beta antibody (e.g., canakinumab or gevokizumab) may be administered to a subject with sickle cell disease at about about 500 mg to about 600 mg in combination with the antibody that specifically binds to p selectin (e.g., crizanlizumab) or antigen binding fragment thereof at about 4 to about 7.5 mg/kg, wherein the combination is administered to the subject about every three weeks.

In some embodiments, the anti-interleukin 1 beta antibody (e.g., canakinumab or gevokizumab) may be administered to a subject with sickle cell disease at about 150 mg to about 300 mg, about 200 mg to about 350 mg, about 250 mg to about 400 mg, about 300 mg to about 450 mg, about 350 mg to about 500 mg, about 400 mg to about 550 mg, about or about 500 mg to about 600 mg in combination with the antibody that specifically binds to p selectin (e.g., crizanlizumab) or antigen binding fragment thereof at about 4 to about 7.5 mg/kg, wherein the combination is administered to the subject about every four weeks. In some embodiments, the anti-interleukin 1 beta antibody (e.g., canakinumab or gevokizumab) may be administered to a subject with sickle cell disease at about 150 mg to about 300 mg in combination with the antibody that specifically binds to p selectin (e.g., crizanlizumab) or antigen binding fragment thereof at about 4 to about 7.5 mg/kg, wherein the combination is administered to the subject about every four weeks. In some embodiments, the anti-interleukin 1 beta antibody (e.g., canakinumab or gevokizumab) may be administered to a subject with sickle cell disease at about about 200 mg to about 350 mg in combination with the antibody that specifically binds to p selectin (e.g., crizanlizumab) or antigen binding fragment thereof at about 4 to about 7.5 mg/kg, wherein the combination is administered to the subject about every four weeks. In some embodiments, the anti-interleukin 1 beta antibody (e.g., canakinumab or gevokizumab) may be administered to a subject with sickle cell disease at about about 250 mg to about 400 mg in combination with the antibody that specifically binds to p selectin (e.g., crizanlizumab) or antigen binding fragment thereof at about 4 to about 7.5 mg/kg, wherein the combination is administered to the subject about every four weeks. In some embodiments, the anti-interleukin 1 beta antibody (e.g., canakinumab or gevokizumab) may be administered to a subject with sickle cell disease at about about 300 mg to about 450 mg in combination with the antibody that specifically binds to p selectin (e.g., crizanlizumab) or antigen binding fragment thereof at about 4 to about 7.5 mg/kg, wherein the combination is administered to the subject about every four weeks. In some embodiments, the anti-interleukin 1 beta antibody (e.g., canakinumab or gevokizumab) may be administered to a subject with sickle cell disease at about about 350 mg to about 500 mg in combination with the antibody that specifically binds to p selectin (e.g., crizanlizumab) or antigen binding fragment thereof at about 4 to about 7.5 mg/kg, wherein the combination is administered to the subject about every four weeks. In some embodiments, the anti-interleukin 1 beta antibody (e.g., canakinumab or gevokizumab) may be administered to a subject with sickle cell disease at about about 400 mg to about 550 mg in combination with the antibody that specifically binds to p selectin (e.g., crizanlizumab) or antigen binding fragment thereof at about 4 to about 7.5 mg/kg, wherein the combination is administered to the subject about every four weeks. In some embodiments, the anti-interleukin 1 beta antibody (e.g., canakinumab or gevokizumab) may be administered to a subject with sickle cell disease at about about 500 mg to about 600 mg in combination with the antibody that specifically binds to p selectin (e.g., crizanlizumab) or antigen binding fragment thereof at about 4 to about 7.5 mg/kg, wherein the combination is administered to the subject about every four weeks.

As a set of non-limiting examples, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4 mg/kg, about 4.5 mg/kg, about 5 mg/kg, about 5.5 mg/kg, about 6 mg/kg, about 6.5 mg/kg, about 7 mg/kg, or about 5 mg/kg. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every two weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every three weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every four weeks. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every five weeks.

In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4 mg/kg every two weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4 mg/kg every three weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4 mg/kg every four weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4 mg/kg every five weeks.

In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4.5 mg/kg every two weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4.5 mg/kg every three weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4.5 mg/kg every four weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4.5 mg/kg every five weeks.

In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5 mg/kg every two weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5 mg/kg every three weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5 mg/kg every four weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5 mg/kg every five weeks.

In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5.5 mg/kg every two weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5.5 mg/kg every three weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5.5 mg/kg every four weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 5.5 mg/kg every five weeks.

In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 6 mg/kg every two weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 6 mg/kg every three weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 6 mg/kg every four weeks. In some embodiments, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 6 mg/kg every five weeks.

As a set of non-limiting examples, the anti-interleukin 1 beta antibody (e.g., canakinumab or gevokizumab) or antigen binding fragment thereof may be administered to a subject with sickle cell disease at 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about or about 600 mg in combination with the antibody that specifically binds to p selectin (e.g., crizanlizumab) or antigen binding fragment thereof at about 4 mg/kg, about 4.5 mg/kg, about 5 mg/kg, about 5.5 mg/kg, about 6 mg/kg, about 6.5 mg/kg, or about 7 mg/kg. In some embodiments, the anti-interleukin 1 beta antibody (e.g., canakinumab or gevokizumab) may be administered to the subject in combination with the antibody that specifically binds to p selectin (e.g., crizanlizumab) or antigen binding fragment thereof every two weeks.

In some embodiments, the anti-interleukin 1 beta antibody (e.g., canakinumab or gevokizumab) or antigen binding fragment thereof may be administered to a subject with sickle cell disease at about 200 mg to about 400 mg in combination with the antibody that specifically binds to p selectin (e.g., crizanlizumab) or antigen binding fragment thereof at about 2.5 mg/kg to about 5 mg/kg every two weeks. In some embodiments, the anti-interleukin 1 beta antibody (e.g., canakinumab or gevokizumab) or antigen binding fragment thereof may be administered to a subject with sickle cell disease at about 200 mg to about 400 mg in combination with the antibody that specifically binds to p selectin (e.g., crizanlizumab) or antigen binding fragment thereof at about 2.5 mg/kg to about 5 mg/kg every three weeks. In some embodiments, the anti-interleukin 1 beta antibody (e.g., canakinumab or gevokizumab) or antigen binding fragment thereof may be administered to a subject with sickle cell disease at about 200 mg to about 400 mg in combination with the antibody that specifically binds to p selectin (e.g., crizanlizumab) or antigen binding fragment thereof at about 2.5 mg/kg to about 5 mg/kg every four weeks. In some embodiments, the anti-interleukin 1 beta antibody (e.g., canakinumab or gevokizumab) or antigen binding fragment thereof may be administered to a subject with sickle cell disease at about 200 mg to about 400 mg in combination with the antibody that specifically binds to p selectin (e.g., crizanlizumab) or antigen binding fragment thereof at about 2.5 mg/kg to about 5 mg/kg every five weeks.

In some embodiments, the anti-interleukin 1 beta antibody (e.g., canakinumab or gevokizumab) or antigen binding fragment thereof may be administered to a subject with sickle cell disease at about 200 mg in combination with the antibody that specifically binds to p selectin (e.g., crizanlizumab) or antigen binding fragment thereof at about 2.5 mg/kg to about 5 mg/kg every two weeks. In some embodiments, the anti-interleukin 1 beta antibody (e.g., canakinumab or gevokizumab) or antigen binding fragment thereof may be administered to a subject with sickle cell disease at about 200 mg in combination with the antibody that specifically binds to p selectin (e.g., crizanlizumab) or antigen binding fragment thereof at about 2.5 mg/kg to about 5 mg/kg every three weeks. In some embodiments, the anti-interleukin 1 beta antibody (e.g., canakinumab or gevokizumab) or antigen binding fragment thereof may be administered to a subject with sickle cell disease at about 200 mg in combination with the antibody that specifically binds to p selectin (e.g., crizanlizumab) or antigen binding fragment thereof at about 2.5 mg/kg to about 5 mg/kg every four weeks. In some embodiments, the anti-interleukin 1 beta antibody (e.g., canakinumab or gevokizumab) or antigen binding fragment thereof may be administered to a subject with sickle cell disease at about 200 mg in combination with the antibody that specifically binds to p selectin (e.g., crizanlizumab) or antigen binding fragment thereof at about 2.5 mg/kg to about 5 mg/kg every five weeks.

In some embodiments, the anti-interleukin 1 beta antibody (e.g., canakinumab or gevokizumab) or antigen binding fragment thereof may be administered to a subject with sickle cell disease at about 250 mg in combination with the antibody that specifically binds to p selectin (e.g., crizanlizumab) or antigen binding fragment thereof at about 2.5 mg/kg to about 5 mg/kg every two weeks. In some embodiments, the anti-interleukin 1 beta antibody (e.g., canakinumab or gevokizumab) or antigen binding fragment thereof may be administered to a subject with sickle cell disease at about 250 mg in combination with the antibody that specifically binds to p selectin (e.g., crizanlizumab) or antigen binding fragment thereof at about 2.5 mg/kg to about 5 mg/kg every three weeks. In some embodiments, the anti-interleukin 1 beta antibody (e.g., canakinumab or gevokizumab) or antigen binding fragment thereof may be administered to a subject with sickle cell disease at about 250 mg in combination with the antibody that specifically binds to p selectin (e.g., crizanlizumab) or antigen binding fragment thereof at about 2.5 mg/kg to about 5 mg/kg every four weeks. In some embodiments, the anti-interleukin 1 beta antibody (e.g., canakinumab or gevokizumab) or antigen binding fragment thereof may be administered to a subject with sickle cell disease at about 250 mg in combination with the antibody that specifically binds to p selectin (e.g., crizanlizumab) or antigen binding fragment thereof at about 2.5 mg/kg to about 5 mg/kg every five weeks.

In some embodiments, the anti-interleukin 1 beta antibody (e.g., canakinumab or gevokizumab) or antigen binding fragment thereof may be administered to a subject with sickle cell disease at about 300 mg in combination with the antibody that specifically binds to p selectin (e.g., crizanlizumab) or antigen binding fragment thereof at about 2.5 mg/kg to about 5 mg/kg every two weeks. In some embodiments, the anti-interleukin 1 beta antibody (e.g., canakinumab or gevokizumab) or antigen binding fragment thereof may be administered to a subject with sickle cell disease at about 300 mg in combination with the antibody that specifically binds to p selectin (e.g., crizanlizumab) or antigen binding fragment thereof at about 2.5 mg/kg to about 5 mg/kg every three weeks. In some embodiments, the anti-interleukin 1 beta antibody (e.g., canakinumab or gevokizumab) or antigen binding fragment thereof may be administered to a subject with sickle cell disease at about 300 mg in combination with the antibody that specifically binds to p selectin (e.g., crizanlizumab) or antigen binding fragment thereof at about 2.5 mg/kg to about 5 mg/kg every four weeks. In some embodiments, the anti-interleukin 1 beta antibody (e.g., canakinumab or gevokizumab) or antigen binding fragment thereof may be administered to a subject with sickle cell disease at about 300 mg in combination with the antibody that specifically binds to p selectin (e.g., crizanlizumab) or antigen binding fragment thereof at about 2.5 mg/kg to about 5 mg/kg every five weeks.

In some embodiments, the anti-interleukin 1 beta antibody (e.g., canakinumab or gevokizumab) or antigen binding fragment thereof may be administered to a subject with sickle cell disease at about 350 mg in combination with the antibody that specifically binds to p selectin (e.g., crizanlizumab) or antigen binding fragment thereof at about 2.5 mg/kg to about 5 mg/kg every two weeks. In some embodiments, the anti-interleukin 1 beta antibody (e.g., canakinumab or gevokizumab) or antigen binding fragment thereof may be administered to a subject with sickle cell disease at about 350 mg in combination with the antibody that specifically binds to p selectin (e.g., crizanlizumab) or antigen binding fragment thereof at about 2.5 mg/kg to about 5 mg/kg every three weeks. In some embodiments, the anti-interleukin 1 beta antibody (e.g., canakinumab or gevokizumab) or antigen binding fragment thereof may be administered to a subject with sickle cell disease at about 350 mg in combination with the antibody that specifically binds to p selectin (e.g., crizanlizumab) or antigen binding fragment thereof at about 2.5 mg/kg to about 5 mg/kg every four weeks. In some embodiments, the anti-interleukin 1 beta antibody (e.g., canakinumab or gevokizumab) or antigen binding fragment thereof may be administered to a subject with sickle cell disease at about 350 mg in combination with the antibody that specifically binds to p selectin (e.g., crizanlizumab) or antigen binding fragment thereof at about 2.5 mg/kg to about 5 mg/kg every five weeks.

In some embodiments, the anti-interleukin 1 beta antibody (e.g., canakinumab or gevokizumab) or antigen binding fragment thereof may be administered to a subject with sickle cell disease at about 400 mg in combination with the antibody that specifically binds to p selectin (e.g., crizanlizumab) or antigen binding fragment thereof at about 2.5 mg/kg to about 5 mg/kg every two weeks. In some embodiments, the anti-interleukin 1 beta antibody (e.g., canakinumab or gevokizumab) or antigen binding fragment thereof may be administered to a subject with sickle cell disease at about 400 mg in combination with the antibody that specifically binds to p selectin (e.g., crizanlizumab) or antigen binding fragment thereof at about 2.5 mg/kg to about 5 mg/kg every three weeks. In some embodiments, the anti-interleukin 1 beta antibody (e.g., canakinumab or gevokizumab) or antigen binding fragment thereof may be administered to a subject with sickle cell disease at about 400 mg in combination with the antibody that specifically binds to p selectin (e.g., crizanlizumab) or antigen binding fragment thereof at about 2.5 mg/kg to about 5 mg/kg every four weeks. In some embodiments, the anti-interleukin 1 beta antibody (e.g., canakinumab or gevokizumab) or antigen binding fragment thereof may be administered to a subject with sickle cell disease at about 400 mg in combination with the antibody that specifically binds to p selectin (e.g., crizanlizumab) or antigen binding fragment thereof at about 2.5 mg/kg to about 5 mg/kg every five weeks.

In some embodiments, the anti-interleukin 1 beta antibody (e.g., canakinumab or gevokizumab) may be combined with L-glutamine (e.g., wherein the L-glutamine is administered to the patient at about 5 mg/day, about 6 mg/day, about 7 mg/day, about 8 mg/day, about 9 mg/day, about 10 mg/day, about 11 mg/day, about 12 mg/day, about 13 mg/day, about 14 mg/day, or 15 mg/day). In some embodiments, any of the described administration regimens for the anti-interleukin 1 beta antibody (e.g., canakinumab or gevokizumab) may be combined with administration of L-glutamine at about 5 mg/day. In some embodiments, any of the described administration regimens for the anti-interleukin 1 beta antibody (e.g., canakinumab or gevokizumab) may be combined with administration of L-glutamine at about 6 mg/day. In some embodiments, any of the described administration regimens for the anti-interleukin 1 beta antibody (e.g., canakinumab or gevokizumab) may be combined with administration of L-glutamine at about 7 mg/day. In some embodiments, any of the described administration regimens for the anti-interleukin 1 beta antibody (e.g., canakinumab or gevokizumab) may be combined with administration of L-glutamine at about 8 mg/day. In some embodiments, any of the described administration regimens for the anti-interleukin 1 beta antibody (e.g., canakinumab or gevokizumab) may be combined with administration of L-glutamine at about 9 mg/day. In some embodiments, any of the described administration regimens for the anti-interleukin 1 beta antibody (e.g., canakinumab or gevokizumab) may be combined with administration of L-glutamine at about 10 mg/day. In some embodiments, any of the described administration regimens for the anti-interleukin 1 beta antibody (e.g., canakinumab or gevokizumab) may be combined with administration of L-glutamine at about 11 mg/day. In some embodiments, any of the described administration regimens for the anti-interleukin 1 beta antibody (e.g., canakinumab or gevokizumab) may be combined with administration of L-glutamine at about 12 mg/day. In some embodiments, any of the described administration regimens for the anti-interleukin 1 beta antibody (e.g., canakinumab or gevokizumab) may be combined with administration of L-glutamine at about 13 mg/day. In some embodiments, any of the described administration regimens for the anti-interleukin 1 beta antibody (e.g., canakinumab or gevokizumab) may be combined with administration of L-glutamine at about 14 mg/day. In some embodiments, any of the described administration regimens for the anti-interleukin 1 beta antibody (e.g., canakinumab or gevokizumab) may be combined with administration of L-glutamine at about 15 mg/day.

Accordingly, any of the methods of treating, preventing, reducing, or eliminating a manifestation or complication associated with sickle cell disease described herein can further include administering a second agent to the subject in need of treatment.

The term “combination” refers to either a fixed combination in one dosage unit form, or a combined administration where an anti-interleukin 1 beta antibody (e.g., canakinumab or gevokizumab) or antigen binding fragment thereof and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration and/or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound of the present invention and a combination partner, are both administered to a patient simultaneously in the form of a single entity or dosage. The term “non-fixed combination” means that the therapeutic agents, e.g., a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g., synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

Pharmaceutical Compositions, Dosages, and Methods of Administration

Also provided herein are compositions, e.g., pharmaceutical compositions, for use in treatment of sickle cell disease. Such compositions include an anti-interleukin 1 beta antibody (e.g., canakinumab or gevokizumab) or antigen binding fragment thereof and may include a pharmaceutically acceptable carrier. Such compositions can further include another agent, e.g., a current standard of care for sickle cell disease.

Pharmaceutical compositions typically include a pharmaceutically acceptable carrier. As used herein the term “pharmaceutically acceptable carrier” refers to a carrier or a diluent that does not cause significant irritation to a subject and does not abrogate the biological activity and properties of the administered anti-interleukin 1 beta antibody (e.g., canakinumab or gevokizumab) or antigen binding fragment thereof and/or any additional therapeutic agent in the composition. Pharmaceutically acceptable carriers may enhance or stabilize the composition or can be used to facilitate preparation of the composition. Pharmaceutically acceptable carriers may include saline, solvents, dispersion media, coatings, antibacterial and antifungal agents, isotonic and absorption delaying agents, and the like, compatible with pharmaceutical administration. An adjuvant may also be included in any of these formulations. Pharmaceutical compositions are typically formulated to be compatible with its intended route of administration. Examples of routes of administration include parenteral (e.g., intravenous, intraarterial, intraperitoneal), oral, intracranial, intrathecal, or intranasal (e.g., inhalation), intradermal, subcutaneous, or transmucosal administration. The phrases “physiologically acceptable carrier” and “pharmaceutically acceptable carrier” may be used interchangeably.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

A pharmaceutical composition of the present disclosure can be administered by a variety of methods known in the art. The route and/or mode of administration may vary depending upon the desired results. In some embodiments, the administration is intravitreal, intravenous, intramuscular, intraperitoneal, or subcutaneous. The pharmaceutically acceptable carrier should be suitable for intravitreal, intravenous, intramuscular, subcutaneous, parenteral, spinal, or epidermal administration (e.g., by injection or infusion). Depending on the route of administration, the active compound(s), i.e., the anti-interleukin 1 beta antibodies (e.g., canakinumab or gevokizumab) and antigen binding fragments optionally in combination with one or more additional therapeutic agent(s) (e.g., any additional therapeutic agent described herein, including, e.g., hydroxyurea and/or crizanlizumab), may be coated in a material to protect the compound(s) from the action of acids and other natural conditions that may inactivate the compound(s).

Typically, a therapeutically effective dose or efficacious dose of the anti-interleukin 1 beta antibodies (e.g., canakinumab or gevokizumab) and antigen binding fragments optionally in combination with one or more additional therapeutic agent(s) (e.g., any additional therapeutic agent described herein, including, e.g., hydroxyurea and/or crizanlizumab) is employed in the pharmaceutical compositions of the present disclosure. Such therapeutic amounts may be formulated into pharmaceutically acceptable dosage forms by conventional methods known to those of skill in the art.

Methods of formulating suitable pharmaceutical compositions are known in the art, see, e.g., Remington: The Science and Practice of Pharmacy. 21^{st} ed., 2005; and the books in the series Drugs and the Pharmaceutical Sciences: a Series of Textbooks and Monographs (Dekker, NY), the contents of each of which are incorporated by reference herein for this purpose. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Dosage regimens for anti-interleukin 1 beta antibody (e.g., canakinumab or gevokizumab) or antigen binding fragment thereof with or without one or more additional therapeutic agent(s) (e.g., any additional therapeutic agent described herein, including, e.g., hydroxyurea and/or crizanlizumab) may be adjusted to provide the optimum desired response (e.g., a therapeutic response). For example, a single bolus of one, two, or additional agents may be administered at one time, several divided doses may be administered over a predetermined period of time, or the dose of one or both agents may be proportionally reduced or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An “effective amount” is an amount sufficient to effect beneficial or desired results. For example, a therapeutic amount or a therapeutically effective amount is one that achieves the desired therapeutic effect. Such a desired therapeutic effect may be treating, preventing, preventing the onset of, curing, delaying, reducing the severity of and/or frequency of, ameliorating, or eliminating at least one manifestation or complication of a disorder or recurring disorder (e.g., at least one manifestation or complication of sickle cell disease), or prolonging the survival of the patient beyond that expected in the absence of such treatment (e.g., in comparison to the absence of treatment or in comparison to the use of one or more other treatments). Such benefits may be seen in the patient themselves (e.g., by comparing the presence or severity of one or more manifestation(s) or complication(s) in the patient before and after administration of the treatment) or in a patient population (e.g., by comparing the presence or severity of one or more manifestation(s) or complication(s) in a group of patients before and after administration of the treatment). This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The skilled artisan (such as a medical doctor) will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. When applied to an individual active ingredient (e.g., an anti-interleukin 1 beta antibody, e.g., canakinumab or gevokizumab) administered alone, the term refers to that ingredient alone. When applied to a combination, the term refers to combined amounts of the active ingredients that result in the therapeutic effect, whether administered in combination, serially or simultaneously.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Dosage regimens for anti-interleukin 1 beta antibodies (e.g., canakinumab or gevokizumab) and antigen binding fragments alone or in combination with one or more additional therapeutic agent(s) (e.g., any additional therapeutic agent described herein, including, e.g., hydroxyurea and/or crizanlizumab) may be adjusted to provide the optimum desired response (e.g., a therapeutic response). For example, a single bolus of one, two, or additional agents may be administered at one time, several divided doses may be administered over a predetermined period of time, or the dose of one or both agents may be proportionally reduced or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage values for compositions comprising an anti-interleukin 1 beta antibody (e.g., canakinumab or gevokizumab) or antigen binding fragment, and/or any additional therapeutic agent(s) (e.g., any additional therapeutic agent described herein, including, e.g., hydroxyurea and/or crizanlizumab), may be selected based on the unique characteristics of the active compound(s), and the particular therapeutic effect to be achieved. A physician or veterinarian can start doses of the antibodies of the disclosure employed in the pharmaceutical composition at levels lower than that required to achieve the desired therapeutic effect and gradually increase the dosage until the desired effect is achieved. In general, effective doses of the compositions of the present disclosure, for the treatment of obesity or another disorder described herein may vary depending upon many different factors, including means of administration, target site, physiological state of the patient, whether the patient is human or an animal, other medications administered, and whether treatment is prophylactic or therapeutic. The selected dosage level may also depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Treatment dosages may be titrated to optimize safety and efficacy.

Kits

Also provided herein are kits including one or more of the compositions provided herein (e.g., an anti-interleukin 1 beta antibody such as canakinumab or gevokizumab; potentially in combination with one or more additional therapeutic agents such as hydroxyurea and/or crizanlizumab) and instructions for use. Such kits may be at any of the dosage amounts or intervals described herein. Instructions for use can include instructions for diagnosis or treatment of sickle cell disease. Kits as provided herein may be used in accordance with any of the methods described herein. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

The disclosure is further illustrated by the following examples, embodiments, and claims, which are illustrative and are not meant to be further limiting. One skilled in the art will recognize many methods and materials similar or equivalent to those described herein, which could be used in the practice of the described compositions and methods. Such equivalents are within the scope of the present disclosure and claims. The contents of all references, including issued patents and published patent applications, cited throughout this application are hereby incorporated by reference.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1: A Multiple-Dose, Subject- and Investigator-Blinded, Placebo-Controlled, Parallel Design Study to Assess the Efficacy, Safety and Tolerability of ACZ885 in Pediatric and Young Adult Patients with Sickle Cell Anemia

TABLE 1
Protocol synopsis

| | |
|---|---|
| Title | A multiple-dose, subject- and investigator-blinded, placebo-controlled, parallel design |
| | study to assess the efficacy, safety and tolerability of ACZ885 (canakinumab) in |
| | pediatric and young adult patients with sickle cell anemia. |
| Brief | Study of efficacy, safety and tolerability of ACZ885 (canakinumab) in pediatric |
| title | and young adult patients with sickle cell anemia. |
| Sponsor and | Novartis |
| Clinical Trial | Phase II |
| Phase | |
| Intervention type | Drug |
| Study type | Interventional |
| Purpose | This study was designed to assess if inhibition of IL-1β by canakinumab would reduce |
| and | daily pain in association with attenuation of intravascular inflammation in pediatric and |
| rationale | young adult patients with sickle cell anemia, therefore allowing development of the |
| | compound for treatment of this disease population. |
| | A pediatric and young adult population (8-20 years of age) were chosen because the |
| | patients were more likely to be without significant accumulated, SCA-related co- |
| | morbidities and are most likely to benefit from the compound's mechanism of action. |
| | The dose of canakinumab selected for the current proof-of-concept trial is 300 mg |
| | s.c. (4 mg/kg for patients ≤40 kg), monthly. This dosing regimen is predicted to achieve |
| | and maintain suppression of the inflammation over time, allowing for tissue repair and |
| | reductions in pain and crisis frequency. |
| | Stratification was planned for patients at treatment randomization according to presence |
| | or absence of concurrent hydroxyurea use because this standard-of-care therapy has |
| | potential therapeutic benefits against the disease, but its practical use in pediatric |
| | populations remains limited. |
| Primary | To determine the effect of canakinumab versus placebo on daily pain experienced by |
| Objective(s) | sickle cell anemia patients (Reduction of average daily pain VAS over the period of |
| | Week 8 to 12 as compared to baseline levels). |
| Secondary | To determine the duration of effects of canakinumab versus placebo on daily |
| Objectives | pain experienced by SCA patients (Reduction of average daily pain VAS over 4- |
| | week intervals up to Week 24 as compared to baseline levels) |
| | To determine the effect of canakinumab versus placebo on laboratory markers of |
| | inflammation (Week 12 versus baseline of: serum hs-CRP, WBC count, Absolute |
| | counts of blood neutrophils, Absolute counts of blood monocytes). |
| | To determine the effect of canakinumab versus placebo on laboratory and |
| | functional markers of hemolysis. |
| | To determine the effect of canakinumab versus placebo on SCA-related days |
| | missed from school or work |
| | To determine the effect of canakinumab versus placebo on reducing the need for |
| | acute blood transfusion |
| | To assess the safety and tolerability of canakinumab in patients with SCA as |
| | measured by adverse events (AEs), including immunogenicity as indicated by |
| | the presence of anti-drug antibodies |
| | To determine the PK of canakinumab in SCA patients |
| Study | This study is an ambulatory-based study of 24 weeks duration followed by an |
| design | additional 24-week open label phase and is subject- and investigator-blinded, |
| | randomized, placebo-controlled, parallel group, non-confirmatory to assess the |
| | clinical efficacy of canakinumab administered s.c. in 6 injections given 28 days apart |
| | (monthly injections). This study will randomize approximately 60 pediatric and young |
| | adult patients (targeting 48 completers) diagnosed with SCA who experience chronic |
| | or episodic pain, i.e., detectable average daily pain level over a 1-2 week screening |
| | period and ≥2 painful episodes in the past year of likely sickle cell etiology requiring |
| | analgesia and interfering with the patient's normal daily routine. |
| Population | This study will recruit patients, 8 to 20 years of age (both inclusive) with SCA. |
| Key | Male and female subjects, 8-20 years of age (both inclusive) diagnosed with |
| Inclusion | sickle cell anemia (HbSS) or sickle beta⁰ thalassemia (documented by family |
| criteria | studies, or analysis of either hemoglobin or DNA). |
| | Screening hs-CRP level ≥1.0 mg/L |
| | Detectable baseline of background or episodic pain measured by daily e-diary |
| | over 1 to 2 weeks during screening period as defined below: |
| | Average daily pain score ≥1 cm without analgesic use over a period of at least 7 |
| | days and/or, |
| | At least one episode of pain requiring analgesic use during a period of up to 14 |
| | days. |
| | History of ≥2 vaso-occlusive pain episodes in the past year, as defined as pain |
| | with no other, non-sickle cell identifiable cause that requires analgesia and |
| | interferes with the patient's normal daily routine. |
| Key | History of known anaphylaxis or hypersensitivity to canakinumab orany |
| Exclusion | component thereof. |
| criteria | Ongoing or treatment within the past 3 months with red blood cell transfusion |
| | therapy, or have evidence of iron overload requiring chelation therapy. |
| | Transcranial Doppler ultrasound in the past year or at screening demonstrating |
| | velocity in middle or anterior cerebral or internal carotid artery |
| | ≥200 cm/sec. Note, transcranial Doppler (TCD) assessment is only required in |
| | patients for whom the trans-temporal acoustic window is sufficient for imaging |
| | the cranial arteries. |
| | Administration of any other blood products within 3 weeks prior of screening |
| | visit. |
| Study | The dose of canakinumab selected for the current trial is 300 mg |
| treatment | s.c. monthly (4 mg/kg for patients ≤40 kg), |
| Efficacy/PD | Reduction of average daily pain VAS over the period of Week 8 to 12 as |
| assessments | compared to baseline levels |
| | Reduction of average daily pain VAS over 4-week intervals up to Week 24 as |
| | compared to baseline levels |
| | Serum hs-CRP, WBC count, absolute counts of blood neutrophils, absolute |
| | counts of blood monocytes, hemoglobin concentration, reticulocyte count, |
| | haptoglobin, LDH, bilirubin (total, direct and indirect), oxygen percent |
| | saturation (SaO2), TCD (Transcranial Doppler). |
| Key | Adverse events, |
| safety | The number of VOPE (vaso-occlusive pain events) and the number of days |
| assessments | with VOPE |
| Other | Wrist actigraphy during daytime and night time |
| assessments | |
| Data | The post-baseline average daily pain VAS (visual analog scale) will be calculated for |
| analysis | each dose from the start of the dose till the day before the next dose, i.e., in an |
| | averagely 4-week interval (e.g., Week 0-4, Week 4-8, etc., depending on the actual |
| | dosing days). The baseline average pain VAS will be calculated as the average of daily |
| | pain scores from screening up to pre-dosing over a period of at least 7 days. Reduction |
| | from baseline in the average daily pain VAS, i.e., baseline minus post-baseline, will |
| | be analyzed using a Bayesian model for repeated measures using Proc MCMC in SAS. |
| | The model includes baseline average daily pain score as a continuous covariate; |
| | treatment group, time and hydroxyurea use history (Yes/No) as fixed factors. |
| | Interactions of time by treatment group and time by baseline covariates will also be |
| | included in the model. Non-informative priors will be used for the fixed effects and |
| | weakly informative prior, for the covariance. Unstructured covariance structure will |
| | be used and other covariance structure will be investigated if there is convergence |
| | problem. Interaction of hydroxyurea use and treatment group will be explored via |
| | visualizations; if substantial interaction is suspected it will be further explored by |
| | including this interaction term in the whole model. |
| | A comparison of canakinumab 300 mg s.c. versus placebo for the period of Week 8- |
| | 12 is of primary interest. Data up to Week 12 will be included in the primary model. |
| | Bayesian posterior probabilities will be used to assess the following PoC criteria as a |
| | guidance for decision making: |
| | Prob (the reduction of average pain score over the period of Week 8-12 in |
| | canakinumab is greater than Placebo) >90%, and |
| | Prob (the reduction of average pain score over the period of Week 8-12 in |
| | canakinumab is greater than Placebo by 1) >50%. |
| | The target difference of 1 is chosen based on the literature search on the Minimally |
| | Clinical Important Difference (MCID) in pain studies. |
| | At the first interim analysis, one futility rule will be assessed. The criterion is defined |
| | as: |
| | Prob (the reduction of average pain score over the period of Week 8-12 in |
| | canakinumab is less than Placebo) >80%. |
| | Data up to Week 24 will also be analyzed with the same model specifications when |
| | there are at least about 50% completers at Week 24 and also upon the study |
| | completion. Pain data in the open label when available will also be summarized, |
| | visualized and explored in appropriate statistical models, to evaluate the maintenance |
| | of the efficacy in patients randomized to canakinumab group and/or improvements in |
| | patients randomized in placebo group. |
| | Patients who have missed two consecutive dosing or more in the blinded treatment |
| | period or have other protocol deviations severely affecting the evaluation of efficacy |
| | will be excluded in the primary efficacy analysis for the blinded period. This is the |
| | primary PD analysis set. |
| Key words | ACZ885, canakinumab, adolescent, sickle cell anemia |

### Results

A. Patients were Evenly Distributed in the Treatment and Placebo Group with Slightly Higher Baseline Activity in the Canakinumab Group
Patient demographics were well balanced between the two study arms.
There were no discontinuations due to study medication
At baseline, study patients had at elevated markers of systemic inflammation, including high sensitive C-reactive protein (hs-CRP), evidence of pain and fatigue symptoms, and increased transcranial Doppler (TCD) velocities compared to similar-aged, normal health populations.
Importantly, all patients except one placebo arm patient were on background hydroxyurea, SoC therapy for SCA shown to reduce by up to 50% the above inflammatory markers and many of the various disease activity parameters
Baseline levels of disease activity were somewhat higher in the canakinumab treatment arm

TABLE 2
Patient Demographics Well Balanced

| | | Canakinumab | Placebo |
|---|---|---|---|
| | | (n = 25) | (n = 24) |
| Age (y) | Mean (SD) | 15.8 (2.69) | 15.6 (3.28) |
| Age | 8-11 | 2 | 2 |
| groups | 12-17 | 14 | 14 |
| (n) | 18-20 | 9 | 8 |
| Gender (n) | Male | 15 | 13 |
| Race | Black | 12 | 13 |
| (n) | White | 12 | 10 |
| | Other | 1 | 1 |
| Hydroxyurea use (n) | | 25 | 23 |
| 24-weeks completed (n) | | 23 | 19 |
| Discontinuation | Lost to | 0 | 2 |
| reason | follow up | | |
| (n) | Physician | 0 | 1 |
| | decision | | |
| | Patient/guardian | 3 | 1 |
| | decision | | |

TABLE 3
Baseline activity higher in canakinumab arm

| | | Canakinumab | Placebo |
|---|---|---|---|
| | | (n = 25) | (n = 24) |
| hs-CRP | Median | 4.0 | 3.0 |
| (mg/L) | [range] | [0.5, 67.4] | [0.7, 79.5] |
| Daily pain | Median | 3.1 | 2.4 |
| (average VAS) | (SD) | (1.74) | (1.75) |
| Daily fatigue | Mean | 3.7 | 3.0 |
| (average VAS) | (SD) | (1.76) | (1.63) |
| Transcranial | Median | 108.0 | 87.0 |
| Doppler (cm/s) | [range] | [23, 142] | [35, 167] |
| Hemoglobin | Mean | 93.4 | 96.3 |
| (g/L) | (SD) | (75.00) | (14.04) |

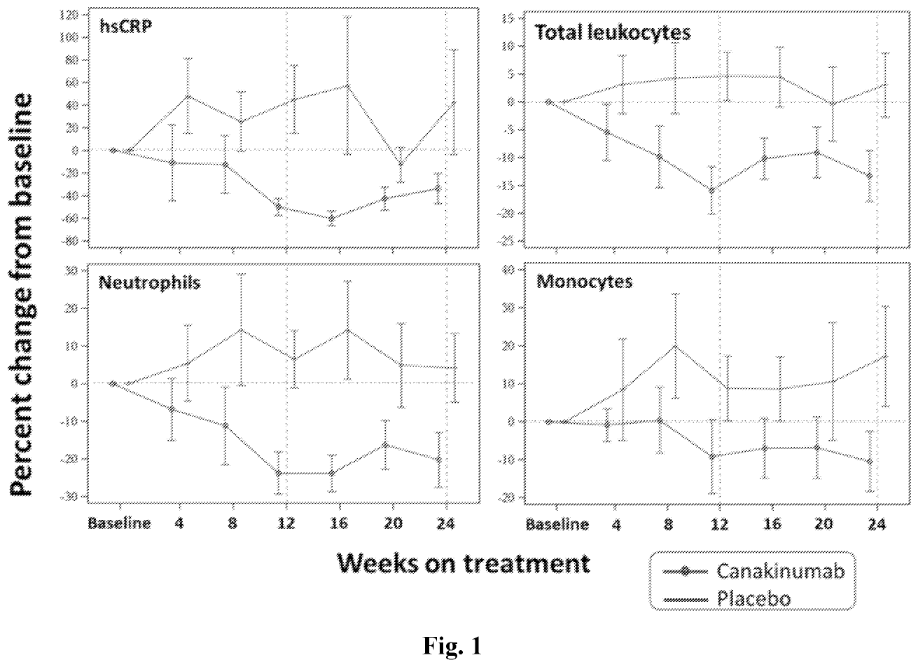

### B. Canakinumab Reduced Systemic Markers of Inflammation (FIG. 1)

SCA is characterized by inflammation that is chronically present, punctuated by periodic flares that cause acute tissue damage.
Literature has suggested that that a major component of SCA inflammation could be driven by a persistent, high rate of hemolysis, with release of RBC contents of heme and other cellular debris activating endothelial receptors of the inflammasome.
Thus, a key study objective was to determine to what degree this sterile inflammation was dependent on IL-lb.
Study results clearly demonstrate that canakinumab-treated pediatric and young adult patients experienced significant reductions in serum markers of systemic inflammation, including hs-CRP and their total circulating leukocyte counts, in particular the absolute counts of neutrophils and monocytes, biomarkers of which have been reported to be predictive of long term morbidity and mortality for this patient population.
Canakinumab effects on the systemic inflammation of these patients appear maximal and most consistent across patients by week 12 and thereafter maintained up to Week 24, the end of the blinded treatment period
Importantly, these anti-inflammatory effects of canakinumab are evident over effects of established HU treatment.

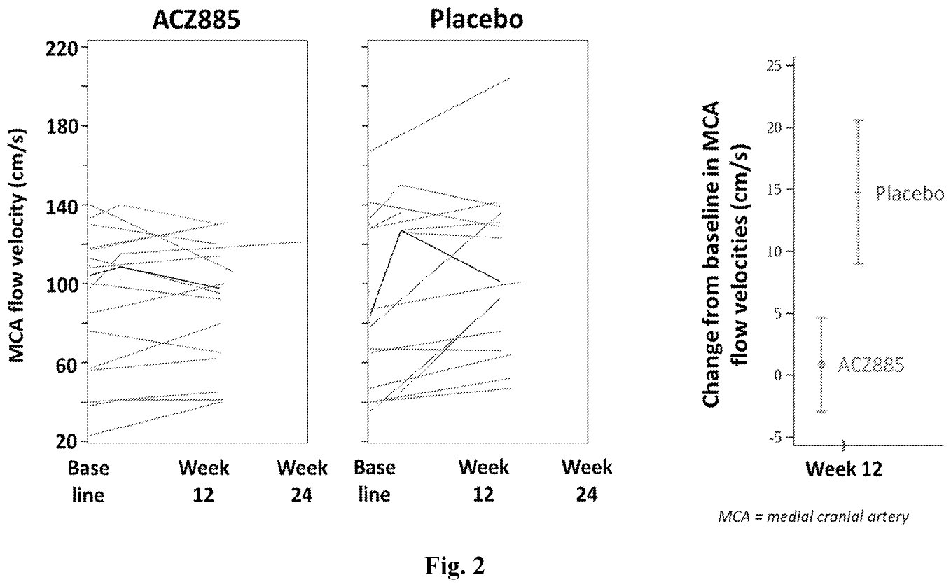

### C. Velocities Reduced Under Canakinumab Compared to Placebo at Week 12 (FIG. 2)

Children with SCA have an increased risk of 250 times over the general pediatric population for cerebral vascular stroke (Behpour et al 2013). Potential contributors to this increased risk in patients include narrowing or occlusion of cerebral vessels, increased viscosity, adherence of RBCs to vascular endothelium, and exhaustion of autoregulatory vasodilation. Transcranial Dopplers are recommended as routine screening in pediatric SCA populations 2-16 years of age to identify those at highest risk, i.e., MCA velocities >200 cm/s. In addition to identifying those at risk for stroke, TCD flow velocities have been correlated with clinical outcomes including measures of cognitive function (Sanchez et al 2010). Finally, inflammatory cytokine serum levels, including IL-1β and IL-IRA, correlate with increased transcranial Doppler flow velocities (Asare et al 2010), and hydroxyurea treatment shows similar clinical benefits and changes in TCD parameters as seen with blood transfusions (Zimmerman et al 2007). Thus, this study used TCD results (historical data over the past year or at screening) to exclude those who have had a TCD measure with flow velocities >200 cm/s. In case of available historical TCD results, all eligible patients should undergo a baseline TCD assessment. To determine the effect of blockade of IL-1β by canakinumab upon TCD velocities, this parameter will be re-assessed at Week 12 of the study.

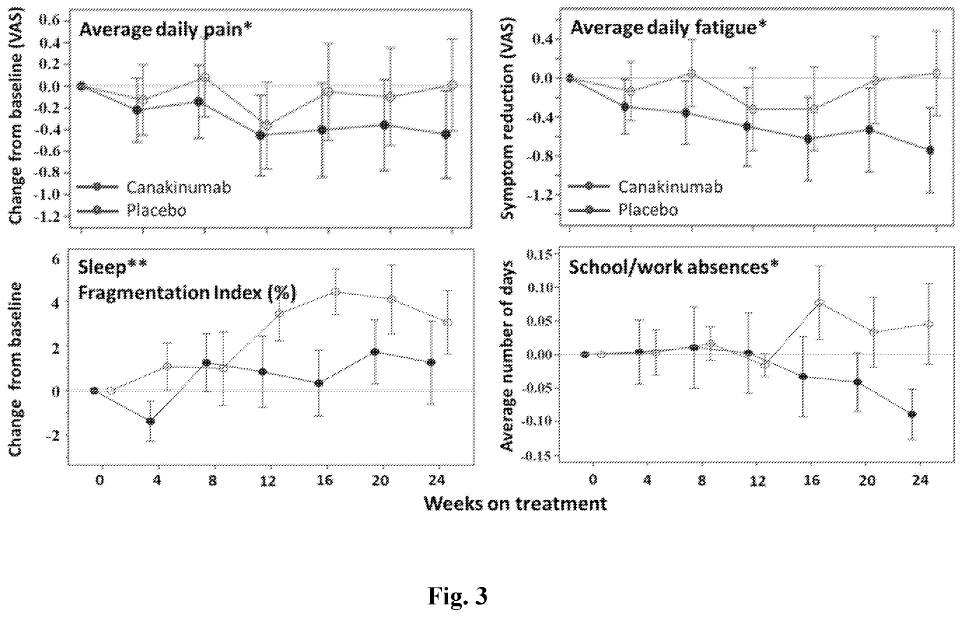

### D. Canakinumab Treated Patients Showed Less Pain & Fatigue, Sleep Better, Less School/Work Absences Vs Placebo Group (FIG. 3)

High daily SCA pain is related to poor nocturnal sleep quality (Valrie et al 2007), and measurement of this outcome is recommended for pediatric acute/recurrent pain clinical trials (McGrath et al 2008). In addition, adolescents with chronic pain tend to withdraw from participation in a wide range of physical activities (Hunfeld et al 2001). Furthermore, a relationship has been shown between pain intensity, mediation use and activity (Rabbitts et al 2014). Wrist actigraphy has been validated in the assessment of sleep and of activity in both healthy adolescents and those with chronic pain through recording motor activity and sleep parameters by means of an electronic device typically worn on the non-dominant wrist (Weiss et al 2010; De Crescenzo et al 2016). The data are typically summarized in epochs of 60 seconds each, with algorithms then used to automatically score sleep and wake periods from the raw activity data generated by these devices. Subsequent analyses will provide activity levels during the daily waking and sleeping periods.
Average daily pain
   Pain diaries are commonly used to assess pain symptoms and response to treatment in children and adolescents with recurrent and chronic pain (McGrath 2008).
   In SCA patients, daily diaries have been shown a more sensitive means of capturing reported pain frequency than through retrospective interviews (Porter 1998). Furthermore, electronic diaries have been shown in adolescents to have additional advantages over the paper format for accuracy as well as compliance in recording patient-reported outcomes (McGrath 2008).
   This study measured pain intensity by eDiary using a simple visual analog scale (VAS); an established means for assessing this symptom (Page 2012; Ruskin 2014). Children with SCA have been shown in numerous studies to reliably self-report pain that is specifically attributable to SCA vaso-occlusion (Shapiro 1995; Dampier 2002; Dampier 2004).
   In this study patients were first screened by eDiary over a 2-week period for a threshold average daily pain intensity of at least 1 on the VAS 11-point numerical rating scale, with 0=no pain and 10=worst pain.
   Patients experiencing pain who respond “yes” for analgesic medication use were instructed to record the category of pain medication (i.e., acetaminophen, NSAID, Narcotic, or Other)
   Average daily pain was reduced in SCA patients treated with canakinumab compared to placebo arm patients, with maximum effects seen over the blinded treatment period after Week 12.
Average daily fatigue
   Similar to pain intensity, a single VAS measure can be used for accurately capturing fatigue data in daily diaries, with convergent validity to validated, multiple-item measures of fatigue (Van Hooff 2007). This 11-point numerical VAS measures fatigue from 0=not at all to 10=extremely.
   Reductions in average daily fatigue were observed in patients receiving canakinumab compared to placebo-treated patients. Early prominent reductions in fatigue occurred a as early as Week 8, and again later within the blinded treatment period at Week 20 and Week 24.
School and work absences
   Daily recording of SCA-related absences from school or work demonstrated reductions in the average number of these days starting after Week 12 for patients treated with canakinumab compared to those receiving placebo. This difference was highly significant by the end of the blinded treatment period at Week 24.
Quality of sleep
   High daily SCA pain is related to poor nocturnal sleep quality (Valrie 2007) and measurement of this outcome is recommended for pediatric trials evaluating outcomes of acute and recurrent pain
   Wrist actigraphy (Phillips Actigraph Watch) has been validated in the assessment of sleep and of activity in both healthy adolescents and those with chronic pain (Weiss 2010; De Crescenzo 2016)
   Patients treated with cankinumab experienced less fragmented sleep, as demonstrated by a reduction in the Sleep Fragmentation Index percentage, defined as the sum of Percent Mobile and Percent Immobile Bouts Less Than 1-Minute Duration to the Number of Immobile Bouts, for the given interval. This is also known as the Index of Restlessness or Movement and Fragmentation Index.
   Calculation of Number of Mobile Bouts and Number of Immobile Bouts 1 Minute in Duration may be performed as follows:
      Number of Mobile Bouts: The total number of continuous blocks, one or more epochs in duration, with each epoch of each block scored as MOBILE, between the Start Time and the End Time of the given interval.
      Number of Immobile Bouts 1 Minute in Duration: The total number of continuous blocks 4 epochs in duration if Epoch Length=15 seconds, 2 epochs in duration if Epoch Length=30 seconds, 1 epoch in duration if Epoch Length=60 seconds (not applicable if Epoch Length is greater than 60 seconds), with each epoch of each block scored as IMMOBILE, between the Start Time and the End Time of the given interval.

### E. Adverse Events

In this study of 49 patients there were 57 SAEs, none of which were related to the study drug
Protocol defined mild, moderate and severe Adverse Events (AEs) and Serious Adverse Events (SAEs), i.e., requiring hospitalization, occurred less often and were distributed among a smaller number of patients in the canakinumab treatment group compared to patients receiving placebo.
There were also a number of severe sequelae associated with SCD crises.
Canakinumab-treated patients had numerical reductions in the number of AEs and SAEs pre-defined as SCA-related.
Moreover these canakinumab-treated patients encountered less sequelae compared to placebo arm patients, including acute chest syndrome, priapism and bone infarction/osteonecrosis.
Finally, as indicated by the annualized hospitalization duration, canakinumab-treated patients spent less time in the hospital compared to placebo patients.
Correspondingly, there was a numerical reduction in median annualized hospitalization duration as measured in days in canakinumab-treated patients compared to patients receiving placebo (0 vs 7.6) as well as the maximum recorded period (37.4 vs. 126.8).

### F. Canakinumab-Treated Group had Less Adverse Events (AEs), Serious Adverse Events (SAEs) and Time Spent in Hospital

TABLE 4
Number of AEs in number of patients

| | Canakinumab | Placebo |
|---|---|---|
| Patients with AEs | 101 in 21 | 146 in 23 |

| | | |
|---|---|---|
| Mild AEs | 68 in 17 | 86 in 18 |
| Moderate AEs | 26 in 15 | 51 in 19 |
| Severe AEs | 7 in 4 | 9 in 3 |

| | | |
|---|---|---|
| Study drug-related AEs | 4 in 3 | 4 in 3 |
| Serious Adverse Events | 19 in 11 | 38 in 15 |
| SAEs per patient | 0.9 | 1.7 |

TABLE 5
Annualized hospitalization duration (days)

| | Canakinumab | Placebo |
|---|---|---|
| Median | 0 | 7.6 |
| Maximum | 37.4 | 126.8 |

TABLE 6
SCA-related SAEs and AEs

| | Canakinumab | Placebo |
|---|---|---|
| Patients with ≥1 SAE | 10 | 12 |
| Pain Crisis (SAE) | 10 | 11 |
| Acute Chest Syndrome (SAE) | 0 | 1 |
| Acute Chest Syndrome (AE) | 1 | 1 |
| Priapism (SAE) | 0 | 1 |
| Priapism (AE) | 0 | 2 |
| Bone infarction (AE) | 0 | 1 |
| Osteonecrosis (SAE) | 0 | 1 |

TABLE 7
Summary of annualized rate of hospitalization (PD analysis)
Has truncated data been used: Truncated due to acute blood transfusion

| | Canakinumab | Placebo |
|---|---|---|
| Statistics | N = 25 | N = 24 |
| Hospitalization reported | | |
| Yes (N %) | 10 (40.0) | 14 (58.3) |
| No (N %) | 15 (60.0) | 10 (41.7) |
| Annualized rate of hospitalization | | |
| N | 25 | 24 |
| mean | 5.65 | 16.32 |
| SD | 10.713 | 27.011 |
| minimum | 0.0 | 0.0 |
| median | 0.00 | 7.56 |
| maximum | 37.4 | 126.8 |

Annualized rate of hospitalization is the number of days hospitalized*365.25/number of days within specified period from day 1

TABLE 8
Overall incidence of AEs - number of events and number of patients (Safety analysis set)
Age group: Adult
Study period: Double-blind period

| | Canakinumab | | Placebo | | Total | |
|---|---|---|---|---|---|---|
| | N = 9 | | N = 8 | | N = 17 | |
| | nE, nS(%) | | nE, nS(%) | | nE, nS(%) | |
| AEs, Patients with AEs | 32, 6 | (66.7) | 62, 8 | (100.0) | 94, 14 | (82.4) |
| AEs of Mild intensity | 14, 3 | (33.3) | 46, 6 | (75.0) | 60, 9 | (52.9) |
| AEs of Moderate intensity | 13, 6 | (66.7) | 16, 8 | (100.0) | 29, 14 | (82.4) |
| AEs of Severe intensity | 5, 3 | (33.3) | 0, 0 | (0.0) | 5, 3 | (17.6) |
| Study drug-related AEs | 2, 1 | (11.1) | 1, 1 | (12.5) | 3, 2 | (11.8) |
| Serious AEs | 8, 4 | (44.4) | 6, 5 | (62.5) | 14, 9 | (52.9) |
| AEs leading to discontinuation | 0, 0 | (0.0) | 0, 0 | (0.0) | 0, 0 | (0.0) |
| of study treatment | | | | | | |
| Study-drug related AEs | 0, 0 | (0.0) | 0, 0 | (0.0) | 0, 0 | (0.0) |
| leading to discontinuation | | | | | | |
| of study treatment | | | | | | |

AEs leading to discontinuation of study treatment are only applicable to the treatment that the patient is on when the AE occurred.
AEs leading to study discontinuation result in a permanent discontinuation of the study.
N = number of patients studied
nE = number of AE events in the category
nS = number of patients with at least one AE in the category
% is based on the number of patients
AEs with incomplete start date are counted only in the total column

TABLE 9
Incidence of sickle cell related AEs by preferred term -
n (percent) of patients (Safety analysis set)
Age group: Allcomers
Study period: Double-blind period

| | Canakinumab | Placebo | Total |
|---|---|---|---|
| | N = 25 | N = 24 | N = 49 |
| | n (%) | n (%) | n (%) |
| Patients with at least one AE | 10 (40.0) | 14 (58.3) | 24 (49.0) |
| Preferred term | | | |
| SICKLE CELL ANAEMIA WITH CRISIS | 10 (40.0) | 11 (45.8) | 21 (42.9) |
| ACUTE CHEST SYNDROME | 1 (4.0) | 2 (8.3) | 3 (6.1) |
| PRIAPISM | 0 (0.0) | 3 (12.5) | 3 (6.1) |
| BONE INFARCTION | 0 (0.0) | 1 (4.2) | 1 (2.0) |
| HAEMOGLOBIN DECREASED | 0 (0.0) | 1 (4.2) | 1 (2.0) |
| OSTEONECROSIS | 0 (0.0) | 1 (4.2) | 1 (2.0) |
| ULTRASOUND DOPPLER ABNORMAL | 0 (0.0) | 1 (4.2) | 1 (2.0) |

TABLE 10
Summary of SAEs (Safety analysis set)
Age group: Allcomers
Study period: Double-blind period

| | Canakinumab | Placebo | Total |
|---|---|---|---|
| Statistics | N = 25 | N = 24 | N = 49 |
| n | 21 | 23 | 44 |
| mean | 0.9 | 1.7 | 1.3 |
| SD | 1.04 | 2.08 | 1.69 |
| minimum | 0 | 0 | 0 |
| median | 1.0 | 1.0 | 1.0 |
| maximum | 3 | 7 | 7 |

n = number of patients with at least one AE within the study period

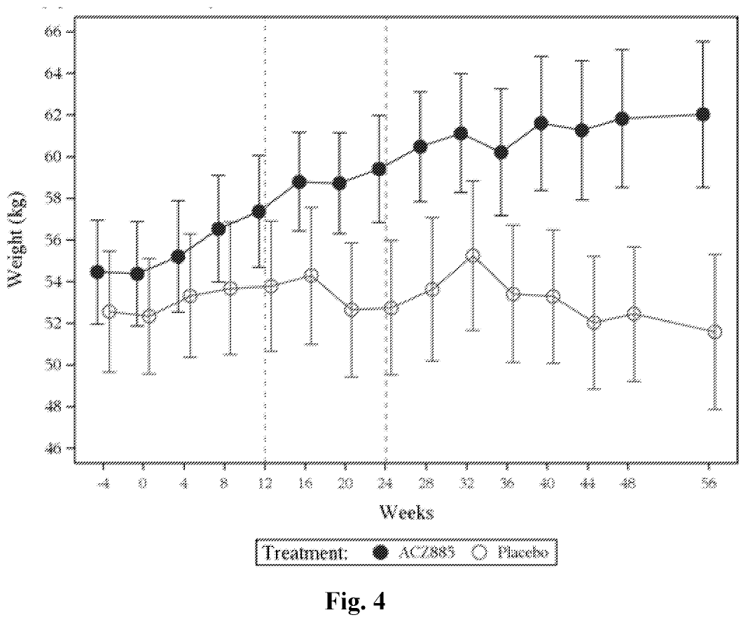

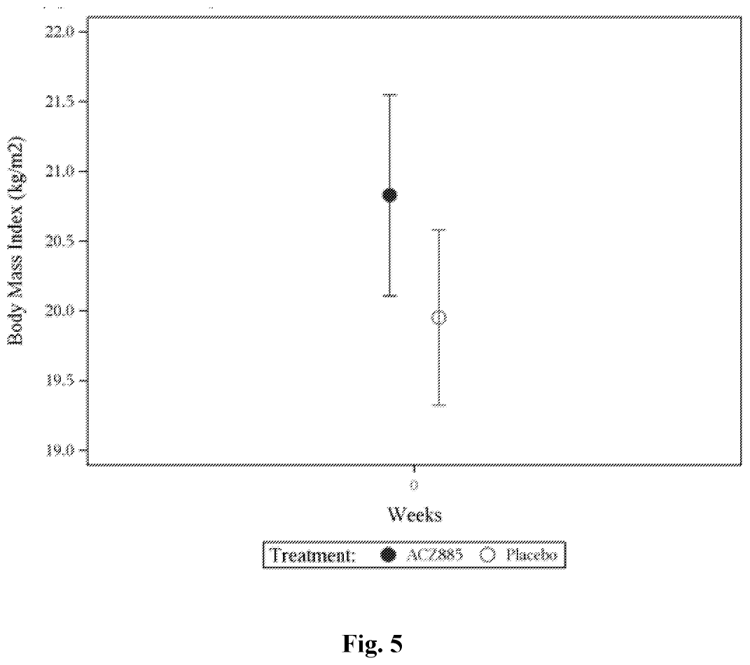

### G. Canakinumab Treated Patients Showed Weight Gain (Increases in Body Weight) Vs Placebo Group (FIGS. 4 and 5)

Low body weight and growth delay are features of sickle cell anemia. Unexpectedly and surprisingly, canakinumab-treated patients in the presently described study showed substantial weight gains throughout the study in comparison to placebo-treated patients. FIG. 4 and FIG. 5 are graphs showing outcome data for patient weight in kg (in FIG. 4) and body mass index (BMI) in kg/m² (FIG. 5) for both placebo treated and canakinumab (ACZ885) treated patients.

### H. Overall Conclusions

Overall, for the Week 24 secondary endpoint, a larger improvement in reduction of pain from baseline was observed for canakinumab compared to Week 12. Canakinumab treatment significantly reduced markers of systemic inflammation compared to placebo subjects over the 24-week blinded treatment period, including hsCRP and circulating leukocytes and neutrophil counts. Transcranial Doppler evaluation at Week 12 showed lower mean and median change from baseline in velocities compared to more highly elevated measures in placebo arm subjects.
Canakinumab-treated subjects also experienced reductions in their average daily fatigue compared to placebo arm subjects, reaching a clinically meaningful difference (>0.5 change in VAS score) by Week 24. Subjects in the canakinumab arm also missed less days of school or work versus placebo arm subjects, starting from Week 16 onwards.
IL-1β blockade by canakinumab did not result in increased infections, but rather reduced the incidence of severe infection over placebo-arm subjects. Overall, the incidence of AEs and SAEs was less in the canakinumab treatment arm compared to the placebo arm (in the double-blind period). Moreover, severe complications of SCD such as priapism, acute chest syndrome, upper respiratory tract infection, spinal edema and avascular necrosis occurred either only in the placebo arm or more frequently in the placebo arm versus the active treatment arm.
In addition, canakinumab-treated subjects also showed substantial weight gains throughout the study course, while the placebo-treated subjects' weight remained comparatively stable over this time. Low body weight is a feature of sickle cell anemia and thus the observed rate of weight gain is a potential indicator of better disease control.

1. A method of treating, preventing, reducing, or eliminating a manifestation or complication associated with sickle cell disease in a subject in need thereof, the method comprising administering to the subject a therapeutically effective amount of an antibody or antigen binding fragment thereof that specifically binds to IL-1 beta, optionally wherein the manifestation or complication is selected from: intravascular inflammation, endothelial dysfunction, pain, fatigue, hospitalization, poor sleep quality, work absences, school absences, narcotic use, acute blood transfusion therapy given for disease severity, chronic blood transfusion therapy given for disease severity, acute chest syndrome, bone infarct, avascular necrosis, osteonecrosis, stroke, cognitive dysfunction, priapism, infarction of penis, a cardiovascular disorder, growth delay, stunted growth, low body mass index (BMI), low body weight, and organ damage.

2. The method of claim 1, wherein the subject is a pediatric patient.

3. The method of claim 1, wherein the subject is an adult patient.

4. The method of any one of claims 1-3, wherein the subject has been diagnosed with sickle cell anemia.

5. The method of any one of the claims 1-4, wherein the subject has HbSS or HbS-beta⁰ thalassemia.

6. The method of any one of the claims 1-5, wherein the subject has an hs-CRP level of at least about 1 mg/L or at least about 2 mg/L prior to administration of the antibody or antigen binding fragment thereof.

7. The method of any one of the claims 1-6, wherein the hs-CRP level of the subject is reduced by at least 20%, at least 30%, or at least 40% after administration of the antibody or antigen binding fragment thereof.

8. The method of any one of the claims 1-6, wherein hs-CRP in the subject is reduced to below about 3 mg/L, to below about 2 mg/L or to below about 1 mg/L after administration of the antibody or antigen binding fragment thereof.

9. The method of any one of claims 1-8, wherein the manifestation or complication associated with sickle cell disease is pain.

10. The method of claim 9, wherein the pain is selected from vaso-occlusive pain events, vaso-occlusive pain crises, and bone pain.

11. The method of claim 9 or 10, wherein the pain is reduced by at least 0.4 in a self-reported pain level scoring from 0 to 10, compared to the average score calculated 1-2 weeks prior to administration of the antibody or antigen binding fragment thereof.

12. A method of reducing pain associated with sickle cell disease in a subject in need thereof, the method comprising administering to the subject a therapeutically effective amount of an antibody or antigen binding fragment that specifically binds to IL-1 beta.

13. The method of any one of claims 1-8, wherein the manifestation or complication associated with sickle cell disease is selected from growth delay, stunted growth, low body mass index (BMI), and/or low body weight.

14. The method of claim 13, wherein the subject in need thereof has an increase in lean body mass, an increase in lean muscle mass, an increase in body mass index (BMI), an increase in body weight, and/or a reversal of growth delay after administration of the antibody or antigen binding fragment that specifically binds to IL-1 beta.

15. The method of claim 13 or 14, wherein the subject has an increase in weight of at least about 5%, at least about 10%, at least about 15%, or at least about 20% in comparison to subject weight prior to administration of the antibody or antigen binding fragment thereof.

16. The method of claim 13 or 14, wherein the subject has an increase in body mass index (BMI) of at least about 5%, at least about 10%, at least about 15%, or at least about 20% in comparison to subject BMI prior to administration of the antibody or antigen binding fragment thereof.

17. The method of claim 13 or 14, wherein the subject has an increase in weight of up to about 5%, up to about 10%, up to about 15%, or up to about 20% in comparison to subject weight prior to administration of the antibody or antigen binding fragment thereof.

18. The method of claim 13 or 14, wherein the subject has an increase in body mass index (BMI) of up to about 5%, up to about 10%, up to about 15%, or up to about 20% in comparison to subject BMI prior to administration of the antibody or antigen binding fragment thereof.

19. The method of any one of claims 13-18, wherein the increase in weight and/or the increase in BMI is measured about 12 weeks, about 24 weeks, about 7 months, about 8 months, about 9 months, about 10 months, about 11 months, about 12 months, about 13 months, or about 14 months after first administration of the antibody or antigen binding fragment thereof.

20. The method of any one of claims 13-18, wherein the increase in weight and/or the increase in BMI is measured at least about 12 weeks, at least about 24 weeks, at least about 7 months, at least about 8 months, at least about 9 months, at least about 10 months, at least about 11 months, at least about 12 months, at least about 13 months, or at least about 14 months after first administration of the antibody or antigen binding fragment thereof.

21. A method of improving growth delay, stunted growth, low body mass index (BMI), and/or low body weight associated with sickle cell disease in a subject in need thereof, the method comprising administering to the subject a therapeutically effective amount of an antibody or antigen binding fragment that specifically binds to IL-1 beta.

22. A method of increasing lean body mass, increasing lean muscle mass, increasing body mass index (BMI), increasing body weight, and/or reversing growth delay in a subject in need thereof, wherein the subject has sickle cell disease, the method comprising administering to the subject a therapeutically effective amount of an antibody or antigen binding fragment that specifically binds to IL-1 beta.

23. The method of any one of claims 1-22, wherein the manifestation or complication associated with sickle cell disease is hospitalization.

24. The method of claim 23, wherein the need of hospitalization is reduced by either (i) annual frequency of hospitalization and/or (ii) duration of hospitalization measured by the number of days annually.

25. The method of claim 24, wherein the annual frequency of hospitalization is reduced by 50%, compared to the frequency in the last 12 months prior to the administration of the antibody or antigen binding fragment thereof.

26. The method of claim 24, wherein the duration of hospitalization measured by the number of days annually is reduced by 50% compared to the average duration of hospitalization in the last 12 months prior to the after administration of the antibody or antigen binding fragment thereof.

27. The method of any one of claims 1-26, wherein the manifestation or complication associated with sickle cell disease is the use of one or more narcotic analgesic agents by the subject.

28. The method of claim 27, wherein the annual use of one or more narcotic analgesic agents by the subject is reduced by at least about 20%, by at least about 30%, by at least about 40%, or by at least about 50% as compared to the use in the last 12 months prior to administration of the antibody or antigen binding fragment thereof.

29. The method of any one of claims 1-28, wherein the manifestation or complication associated with sickle cell disease is acute and/or chronic blood transfusion therapy for treatment of anemia.

30. The method of claim 29, wherein the total amount of transfused blood is reduced by at least about 20%, by at least about 30%, by at least about 40%, or by at least about 50% as compared to the amount of blood transfused in the last 12 months prior to administration of the antibody or antigen binding fragment thereof.

31. The method of any one of claims 1-30, wherein the manifestation or complication associated with sickle cell disease is organ damage.

32. The method of claim 31, wherein the organ damage is end organ damage.

33. The method of claim 31 or claim 32, where the organ damage is damage to one or more organs selected from: spleen, brain, eyes, lungs, liver, heart, kidneys, penis, joints, bones, and skin.

34. A method of preventing organ damage associated with sickle cell disease in a subject in need thereof, the method comprising administering to the subject a therapeutically effective amount of an antibody or antigen binding fragment that specifically binds to IL-1 beta.

35. The method of any one of the claims 31 to 34, wherein the organ damage is sickle cell disease associated nephropathy.

36. The method of any one of claims 1-35, wherein the manifestation or complication associated with sickle cell disease is stroke, optionally wherein the stroke is selected from the group consisting of an ischemic stroke or a hemorrhagic stroke.

37. A method of preventing stroke associated with sickle cell disease in a subject in need thereof, the method comprising administering to the subject a therapeutically effective amount of an antibody or antigen binding fragment that specifically binds to IL-1 beta.

38. The method of any one of the preceding claims, wherein the antibody or antigen binding fragment that specifically binds to IL-1 beta is administered intravenously (i.v.).

39. The method of any one of the preceding claims, wherein the antibody or antigen binding fragment that specifically binds to IL-1 beta is administered subcutaneously (s.c.).

40. The method of any one of the preceding claims, wherein the antibody or antigen binding fragment that specifically binds to IL-1 beta is canakinumab.

41. The method of any one of the claims 1 to 39, wherein the antibody or antigen binding fragment that specifically binds to IL-1 beta is gevokizumab.

42. The method of any one of the preceding claims, wherein the antibody or antigen binding fragment that specifically binds to IL-1 beta is administered to the patient at about 30 mg to about 600 mg.

43. The method of any one of claims 1-42, wherein the antibody or antigen binding fragment that specifically binds to IL-1 beta is administered to the patient every two weeks, every three weeks, every four weeks, every six weeks, every eight weeks, every nine weeks, or every twelve weeks.

44. The method of any one of the preceding claims, wherein about 150 mg to about 400 mg canakinumab is administered to the patient every four weeks, every six weeks, every eight weeks, or every twelve weeks.

45. The method of any one of the preceding claims, wherein canakinumab is administered to the patient at about 150 mg every two weeks, every three weeks, every four weeks, every eight weeks, or every twelve weeks.

46. The method of any one of the preceding claims, wherein canakinumab is administered to the patient at about 200 mg every two weeks, every three weeks, every four weeks, every eight weeks, or every twelve weeks.

47. The method of any one of the preceding claims, wherein canakinumab is administered to the patient at about 250 mg every two weeks, every three weeks, every four weeks, every eight weeks, or every twelve weeks.

48. The method of any one of the preceding claims, wherein canakinumab is administered to the patient at about 300 mg every two weeks, every three weeks, every four weeks, every eight weeks, or every twelve weeks.

49. The method of any one of the preceding claims, wherein canakinumab is administered to the patient at about 400 mg every two weeks, every three weeks, every four weeks, every eight weeks, or every twelve weeks.

50. The method of any one of the preceding claims, wherein the subject is administered a therapeutically effective amount of an additional therapeutic agent.

51. The method of claim 50, wherein the additional therapeutic agent is selected from an antibody or antigen binding fragment thereof that specifically binds to p selectin, L-glutamine oral powder, and/or an agent that increases fetal hemoglobin.

52. The method of claim 50 or 51, wherein the additional therapeutic agent is selected from hydroxyurea, an antibody or antigen binding fragment thereof that specifically binds to p selectin, L-glutamine oral powder, voxelotor, stem cells comprising a lentiviral vector which inserts a functioning version of the HBB gene, and combinations thereof.

53. The method of any one of the claims 51 to 52, wherein the antibody or antigen binding fragment thereof that specifically binds to p selectin is crizanlizumab.

54. The method of any one of the claims 50 to 53, wherein crizanlizumab is administered to the patient at about 2.5 mg/kg, about 5 mg/kg, or about 7.5 mg/kg.

55. The method of any one of claims 50 to 54, wherein crizanlizumab is administered to the patient every four weeks, optionally wherein the first 2 doses are 2 weeks apart.

56. The method of any one of claims 50 to 55, wherein the IL-1 beta binding antibody or antigen binding fragment thereof is administered to the subject in combination with crizanlizumab on the same day, optionally not more than 2 hours apart or not more than one hour apart.

57. The method of any one of the preceding claims, wherein the antibody or antigen binding fragment thereof that specifically binds to IL-1 beta is administered to the subject in a loading phase followed by a maintenance phase, wherein the subject receives a higher amount of the antibody during the loading phase than during the maintenance phase over the same given period of time.

58. The method of claim 57, wherein the loading phase is at least 3 months.

59. The method of claim 57 or 58, wherein the administration interval within the loading phase is the same as that within the maintenance phase.

60. The method of claim 59, wherein the dose within the loading phase (loading dose) is at least twice the amount of the dose within the maintenance phase (maintenance dose).

61. The method of claim 57 or 58, wherein the loading dose is the same as the maintenance dose.

62. The method of claim 61, wherein the administration interval within the maintenance phase is twice or three times as long as the interval within the loading phase.

63. An antibody or antigen binding fragment thereof that specifically binds to IL-1 beta for use in treating one or more manifestations or complications of sickle cell disease, optionally wherein the manifestation or complication is selected from: intravascular inflammation, endothelial dysfunction, pain, fatigue, hospitalization, poor sleep quality, work absences, school absences, narcotic use, acute blood transfusion therapy given for disease severity, chronic blood transfusion therapy given for disease severity, acute chest syndrome, bone infarct, avascular necrosis, osteonecrosis, stroke, cognitive dysfunction, priapism, infarction of penis, a cardiovascular disorder, growth delay, stunted growth, low body mass index (BMI), low body weight, and organ damage.

64. The antibody or antigen binding fragment thereof of claim 63, wherein the subject is a pediatric patient.

65. The antibody or antigen binding fragment thereof of claim 63, wherein the subject is an adult patient.

66. The antibody or antigen binding fragment thereof of any one of claims 63-65, wherein the subject has been diagnosed with sickle cell anemia.

67. The antibody or antigen binding fragment thereof of any one of the claims 63-66, wherein the subject has HbSS or HbS-beta⁰ thalassemia.

68. The antibody or antigen binding fragment thereof of any one of the claims 63-67, wherein the subject has an hs-CRP level of at least about 1 mg/L or at least about 2 mg/L prior to administration of the antibody or antigen binding fragment thereof.

69. The antibody or antigen binding fragment thereof of any one of the claims 63-68, wherein the hs-CRP level of the subject is reduced by at least 20%, at least 30%, or at least 40% after administration of the antibody or antigen binding fragment thereof.

70. The antibody or antigen binding fragment thereof of any one of the claims 63-68, wherein hs-CRP in the subject is reduced to below about 3 mg/L, to below about 2 mg/L or to below about 1 mg/L after administration of the antibody or antigen binding fragment thereof.

71. The antibody or antigen binding fragment thereof of any one of claims 63-70, wherein the manifestation or complication associated with sickle cell disease is pain.

72. The antibody or antigen binding fragment thereof of claim 71, wherein the pain is selected from vaso-occlusive pain events, vaso-occlusive pain crises, and bone pain.

73. The antibody or antigen binding fragment thereof of claim 71 or 72, wherein the pain is reduced by at least 0.4 in a self-reported pain level scoring from 0 to 10, compared to the average score calculated 1-2 weeks prior to administration of the antibody or antigen binding fragment thereof.

74. An antibody or antigen binding fragment thereof that specifically binds to IL-1 beta for use in reducing pain associated with sickle cell disease in a subject in need thereof, the method comprising administering to the subject a therapeutically effective amount of an antibody or antigen binding fragment that specifically binds to IL-1 beta.

75. The antibody or antigen binding fragment thereof of any one of claims 63-70, wherein the manifestation or complication associated with sickle cell disease is selected from growth delay, stunted growth, low body mass index (BMI), and/or low body weight.

76. The antibody or antigen binding fragment thereof of claim 75, wherein the subject in need thereof has an increase in lean body mass, an increase in lean muscle mass, an increase in body mass index (BMI), an increase in body weight, and/or a reversal of growth delay after administration of the antibody or antigen binding fragment that specifically binds to IL-1 beta.

77. The antibody or antigen binding fragment thereof of claim 75 or 76, wherein the subject has an increase in weight of at least about 5%, at least about 10%, at least about 15%, or at least about 20% in comparison to subject weight prior to administration of the antibody or antigen binding fragment thereof.

78. The antibody or antigen binding fragment thereof of claim 75 or 76, wherein the subject has an increase in body mass index (BMI) of at least about 5%, at least about 10%, at least about 15%, or at least about 20% in comparison to subject BMI prior to administration of the antibody or antigen binding fragment thereof.

79. The antibody or antigen binding fragment thereof of claim 75 or 76, wherein the subject has an increase in weight of up to about 5%, up to about 10%, up to about 15%, or up to about 20% in comparison to subject weight prior to administration of the antibody or antigen binding fragment thereof.

80. The antibody or antigen binding fragment thereof of claim 75 or 76, wherein the subject has an increase in body mass index (BMI) of up to about 5%, up to about 10%, up to about 15%, or up to about 20% in comparison to subject BMI prior to administration of the antibody or antigen binding fragment thereof.

81. The antibody or antigen binding fragment thereof of any one of claims 75-80, wherein the increase in weight and/or the increase in BMI is measured about 12 weeks, about 24 weeks, about 7 months, about 8 months, about 9 months, about 10 months, about 11 months, about 12 months, about 13 months, or about 14 months after first administration of the antibody or antigen binding fragment thereof.

82. The antibody or antigen binding fragment thereof of any one of claims 75-80, wherein the increase in weight and/or the increase in BMI is measured at least about 12 weeks, at least about 24 weeks, at least about 7 months, at least about 8 months, at least about 9 months, at least about 10 months, at least about 11 months, at least about 12 months, at least about 13 months, or at least about 14 months after first administration of the antibody or antigen binding fragment thereof.

83. An antibody or antigen binding fragment thereof that specifically binds to IL-1 beta for use in improving growth delay, stunted growth, low body mass index (BMI), and/or low body weight associated with sickle cell disease in a subject in need thereof, the method comprising administering to the subject a therapeutically effective amount of an antibody or antigen binding fragment that specifically binds to IL-1 beta.

84. An antibody or antigen binding fragment thereof that specifically binds to IL-1 beta for use in increasing lean body mass, increasing lean muscle mass, increasing body mass index (BMI), increasing body weight, and/or reversing growth delay in a subject in need thereof, wherein the subject has sickle cell disease, the method comprising administering to the subject a therapeutically effective amount of an antibody or antigen binding fragment that specifically binds to IL-1 beta.

85. The antibody or antigen binding fragment thereof of any one of claims 63-84, wherein the manifestation or complication associated with sickle cell disease is hospitalization.

86. The antibody or antigen binding fragment thereof of claim 85, wherein the need of hospitalization is reduced by either (i) annual frequency of hospitalization and/or (ii) duration of hospitalization measured by the number of days annually.

87. The antibody or antigen binding fragment thereof of claim 86, wherein the annual frequency of hospitalization is reduced by 50%, compared to the frequency in the last 12 months prior to the administration of the antibody or antigen binding fragment thereof.

88. The antibody or antigen binding fragment thereof of claim 86, wherein the duration of hospitalization measured by the number of days annually is reduced by 50% compared to the average duration of hospitalization in the last 12 months prior to the after administration of the antibody or antigen binding fragment thereof.

89. The antibody or antigen binding fragment thereof of any one of claims 63-88, wherein the manifestation or complication associated with sickle cell disease is the use of one or more narcotic analgesic agents by the subject.

90. The antibody or antigen binding fragment thereof of claim 89, wherein the annual use of one or more narcotic analgesic agents by the subject is reduced by at least about 20%, by at least about 30%, by at least about 40%, or by at least about 50% as compared to the use in the last 12 months prior to administration of the antibody or antigen binding fragment thereof.

91. The antibody or antigen binding fragment thereof of any one of claims 63-90, wherein the manifestation or complication associated with sickle cell disease is acute and/or chronic blood transfusion therapy for treatment of anemia.

92. The antibody or antigen binding fragment thereof of claim 91, wherein the total amount of transfused blood is reduced by at least about 20%, by at least about 30%, by at least about 40%, or by at least about 50% as compared to the amount of blood transfused in the last 12 months prior to administration of the antibody or antigen binding fragment thereof.

93. The antibody or antigen binding fragment thereof of any one of claims 63-92, wherein the manifestation or complication associated with sickle cell disease is organ damage.

94. The antibody or antigen binding fragment thereof of claim 93, wherein the organ damage is end organ damage.

95. The antibody or antigen binding fragment thereof of claim 93 or claim 94, where the organ damage is damage to one or more organs selected from: spleen, brain, eyes, lungs, liver, heart, kidneys, penis, joints, bones, and skin.

96. An antibody or antigen binding fragment thereof that specifically binds to IL-1 beta for use in treating or preventing organ damage associated with sickle cell disease in a subject in need thereof, the method comprising administering to the subject a therapeutically effective amount of an antibody or antigen binding fragment that specifically binds to IL-1 beta.

97. The antibody or antigen binding fragment thereof of any one of the claims 93-96, wherein the organ damage is sickle cell disease associated nephropathy.

98. The antibody or antigen binding fragment thereof of any one of claims 63-97, wherein the manifestation or complication associated with sickle cell disease is stroke, optionally wherein the stroke is selected from the group consisting of an ischemic stroke or a hemorrhagic stroke.

99. An antibody or antigen binding fragment thereof that specifically binds to IL-1 beta for use in treating or preventing stroke associated with sickle cell disease in a subject in need thereof, the method comprising administering to the subject a therapeutically effective amount of an antibody or antigen binding fragment that specifically binds to IL-1 beta.

100. The antibody or antigen binding fragment thereof of any one of the preceding claims, wherein the antibody or antigen binding fragment that specifically binds to IL-1 beta is administered intravenously (i.v.).

101. The antibody or antigen binding fragment thereof of any one of the preceding claims, wherein the antibody or antigen binding fragment that specifically binds to IL-1 beta is administered subcutaneously (s.c.).

102. The antibody or antigen binding fragment thereof of any one of the preceding claims, wherein the antibody or antigen binding fragment that specifically binds to IL-1 beta is canakinumab.

103. The antibody or antigen binding fragment thereof of any one of the claims 63-101, wherein the antibody or antigen binding fragment that specifically binds to IL-1 beta is gevokizumab.

104. The antibody or antigen binding fragment thereof of any one of the preceding claims, wherein the antibody or antigen binding fragment that specifically binds to IL-1 beta is administered to the patient at about 30 mg to about 600 mg.

105. The antibody or antigen binding fragment thereof of any one of claims 63-104, wherein the antibody or antigen binding fragment that specifically binds to IL-1 beta is administered to the patient every two weeks, every three weeks, every four weeks, every six weeks, every eight weeks, every nine weeks, or every twelve weeks.

106. The antibody or antigen binding fragment thereof of any one of the preceding claims, wherein about 150 mg to about 400 mg canakinumab is administered to the patient every four weeks, every six weeks, every eight weeks, or every twelve weeks.

107. The antibody or antigen binding fragment thereof of any one of the preceding claims, wherein canakinumab is administered to the patient at about 150 mg every two weeks, every three weeks, every four weeks, every eight weeks, or every twelve weeks.

108. The antibody or antigen binding fragment thereof of any one of the preceding claims, wherein canakinumab is administered to the patient at about 200 mg every two weeks, every three weeks, every four weeks, every eight weeks, or every twelve weeks.

109. The antibody or antigen binding fragment thereof of any one of the preceding claims, wherein canakinumab is administered to the patient at about 250 mg every two weeks, every three weeks, every four weeks, every eight weeks, or every twelve weeks.

110. The antibody or antigen binding fragment thereof of any one of the preceding claims, wherein canakinumab is administered to the patient at about 300 mg every two weeks, every three weeks, every four weeks, every eight weeks, or every twelve weeks.

111. The antibody or antigen binding fragment thereof of any one of the preceding claims, wherein canakinumab is administered to the patient at about 400 mg every two weeks, every three weeks, every four weeks, every eight weeks, or every twelve weeks.

112. The antibody or antigen binding fragment thereof of any one of the preceding claims, wherein the subject is administered a therapeutically effective amount of an additional therapeutic agent.

113. The antibody or antigen binding fragment thereof of claim 112, wherein the additional therapeutic agent is selected from an antibody or antigen binding fragment thereof that specifically binds to p selectin, L-glutamine oral powder, and/or an agent that increases fetal hemoglobin.

114. The antibody or antigen binding fragment thereof of claim 112 or 113, wherein the additional therapeutic agent is selected from hydroxyurea, an antibody or antigen binding fragment thereof that specifically binds to p selectin, L-glutamine oral powder, voxelotor, stem cells comprising a lentiviral vector which inserts a functioning version of the HBB gene, and combinations thereof.

115. The antibody or antigen binding fragment thereof of claim 113 or 114, wherein the antibody or antigen binding fragment thereof that specifically binds to p selectin is crizanlizumab.

116. The antibody or antigen binding fragment thereof of any one of the claims 112-115, wherein crizanlizumab is administered to the patient at about 2.5 mg/kg, about 5 mg/kg, or about 7.5 mg/kg.

117. The antibody or antigen binding fragment thereof of any one of claims 112-116, wherein crizanlizumab is administered to the patient every four weeks, optionally wherein the first 2 doses are 2 weeks apart.

118. The antibody or antigen binding fragment thereof of any one of claims 112-117, wherein the IL-1 beta binding antibody or antigen binding fragment thereof is administered to the subject in combination with crizanlizumab on the same day, optionally not more than 2 hours apart or not more than one hour apart.

119. The antibody or antigen binding fragment thereof of any one of the preceding claims, wherein the antibody or antigen binding fragment thereof that specifically binds to IL-1 beta is administered to the subject in a loading phase followed by a maintenance phase, wherein the subject receives a higher amount of the antibody during the loading phase than during the maintenance phase over the same given period of time.

120. The antibody or antigen binding fragment thereof of claim 119, wherein the loading phase is at least 3 months.

121. The antibody or antigen binding fragment thereof of claim 119 or 120, wherein the administration interval within the loading phase is the same as that within the maintenance phase.

122. The antibody or antigen binding fragment thereof of claim 121, wherein the dose within the loading phase (loading dose) is at least twice the amount of the dose within the maintenance phase (maintenance dose).

123. The antibody or antigen binding fragment thereof of claim 119 or 120, wherein the loading dose is the same as the maintenance dose.

124. The antibody or antigen binding fragment thereof of claim 123, wherein the administration interval within the maintenance phase is twice or three times as long as the interval within the loading phase.
